(19) United States
(12) Patent Application Publication
(10) Pub. No.: US 2025/0297935 A1
(43) Pub. Date: Sep. 25, 2025

(54) WAVE INTERFERENCE IN RHEOMETRY

(71) Applicant: HYDRAMOTION LIMITED, Malton, North Yorkshire, Yorkshire (GB)

(72) Inventor: John Gallagher, Malton, North Yorkshire, Yorkshire (GB)

(21) Appl. No.: 18/869,987

(22) PCT Filed: May 26, 2023
(86) PCT No.: PCT/GB2023/051391

§ 371 Date: Nov. 27, 2024

(30) Foreign Application Priority Data
May 27, 2022 (GB) 2207881.0

(51) Int. Cl. G01N 11/10; G01N 11/00

(52) U.S. Cl. CPC G01N 11/10; G01N 2011/0073

(57) ABSTRACT

A method of measuring a material property of a viscoelastic fluid using one or more vibratory transducers, the method comprising: vibrating one or more vibratory transducers in the viscoelastic fluid to generate a first wave propagating from a first surface of the one or more vibratory transducers and a second wave propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference; and determining a material property of the viscoelastic fluid based on the vibrating of the one or more vibratory transducers in the viscoelastic fluid.

### TECHNICAL FIELD

This disclosure relates to the damping of vibrations within fluids, including the use of damping to obtain measurements of physical and rheological properties of materials such as measurements of viscosity.

### BACKGROUND

Physical and rheological properties of a fluid can be measured by applying an oscillatory stimulus to the fluid and observing a fluid mechanical response. From an observed fluid mechanical response (for example, a degree of damping and/or stiffness, and/or a resonant frequency), a measurement of a property of a fluid can be obtained, such as viscosity, density, storage modulus, loss modulus, and loss tangent.

By way of example, a degree of damping may be determined from an amplitude of vibration or a change in amplitude, a resonant frequency or a change in resonant frequency, a rate of decay of vibration, or a quality (Q) factor, or a loss factor, which is the reciprocal of a quality factor.

Resonant viscometers measure viscosity by determining the damping effect that viscous fluids have on a mechanical oscillator immersed in the fluid. The presence of viscosity increases shear stress at the oscillator surface. The shear stress creates a damping force, which dissipates energy from the oscillator. For a mechanical oscillator operating at resonance, this reduces the Q factor at resonance. The Q factor is therefore an inverse indicator of the viscosity. The loss factor is the inverse of the Q factor, and therefore an increase in viscosity causes an increase in the loss factor. Historically, resonant viscometers have been shown to work well (i.e. that their loss factor effectively varies with fluid viscosity) with purely viscous fluids and fluids that are mildly viscoelastic (non-Newtonian fluids) where the tan A (i.e. the loss tangent) is greater than 1.

The loss tangent is given by the following expression: tan Δ=ωμ′/G′, where ω is the angular frequency of oscillation, μ′ is the dynamic viscosity of the fluid, and G′ is the storage modulus of the fluid.

Newtonian fluids are purely viscous, i.e. without any elastic behaviour. There is no storage modulus. The loss tangent, tan Δ, is infinite. Examples of such fluids are water, aqueous solutions, sugar syrups, alcohol, most pure oils, most hydrocarbons, and gases.

Non-Newtonian fluids may be viscoelastic and so have tan Δ<∞. Examples of viscoelastic fluids include blood, suspensions, emulsions, and most synthetic materials. Strongly viscoelastic fluids may have tan Δ<1. Examples of strongly viscoelastic fluids include liquid polymers, polymer melts, rubber solutions, synthetic oils, detergents and foodstuffs.

### SUMMARY

According to a first aspect, there is described method of measuring a material property of a viscoelastic fluid using one or more vibratory transducers, the method comprising: vibrating one or more vibratory transducers in the viscoelastic fluid to generate a first wave propagating from a first surface of the one or more vibratory transducers and a second wave propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces; and determining a material property of the viscoelastic fluid based on the vibrating of the one or more vibratory transducers in the viscoelastic fluid.

According to a further aspect, there is provided a non-transitory computer-readable medium having instructions stored thereon that, when executed by one or more processors of a system comprising one or more vibratory transducers, cause the one or more processors to perform the above-described method.

According to a further aspect, there is described an apparatus for measuring a material property of a viscoelastic fluid using one or more vibratory transducers, the apparatus comprising: one or more vibratory transducers, the one or more vibratory transducers comprising a first surface and a second surface; means for vibrating the one or more vibratory transducers such that, when vibrated in a viscoelastic fluid, a first wave is generated propagating from a first surface of the one or more vibratory transducers and a second wave is generated propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces; and means for determining a material property of the viscoelastic fluid based on the vibrating of the one or more vibratory transducers in the viscoelastic fluid including the net constructive or destructive interference.

### BRIEF DESCRIPTION OF THE DRAWINGS

The invention will be described in more detail by way of example only with reference to the accompanying drawings, in which:

FIG. 1 illustrates a shear wave generated from an oscillating surface in a fluid;

FIG. 2 illustrates a simple model for a viscoelastic fluid as a spring and damper system;

FIG. 3 is a graph of F(Δ)=1/(sin(Δ/2)·\sqrt{2·sin Δ}) plotted against Δ (in units of degrees);

FIG. 4 illustrates shear wave velocity fields generated by a primary oscillating surface (i.e. detector) and a secondary oscillating surface (i.e. irradiator) for a first viscoelastic fluid that has relatively low μ′ and relatively low G′;

FIG. 5 illustrates shear wave velocity fields generated by a primary oscillating surface (i.e. detector) and a secondary oscillating surface (i.e. irradiator) for a second viscoelastic fluid that has relatively high μ′ and relatively high G′;

FIG. 6 illustrates the propagation of a shear wave from a surface having a radius of curvature undergoing torsional vibration; and

FIGS. 7 to 47 illustrate configurations for vibratory transducers in accordance with the techniques of this disclosure;

FIG. 48 illustrates an example elongate member in which geometric damping may arise during its vibration;

FIG. 49 illustrates further example elongate members in which geometric damping may arise during their vibration;

FIG. 50 is a graph of damping factor and penetration depth against damping factor showing three regimes of varying degrees of linearity;

FIG. 51 illustrates acoustic wave generation from the transverse vibration of an elongate member in a fluid;

FIG. 52 illustrates shear wave generation from the transverse vibration of an elongate member during laminar flow; and

FIG. 53 illustrates a flow chart of a method in accordance with the techniques of this disclosure.

### DETAILED DESCRIPTION

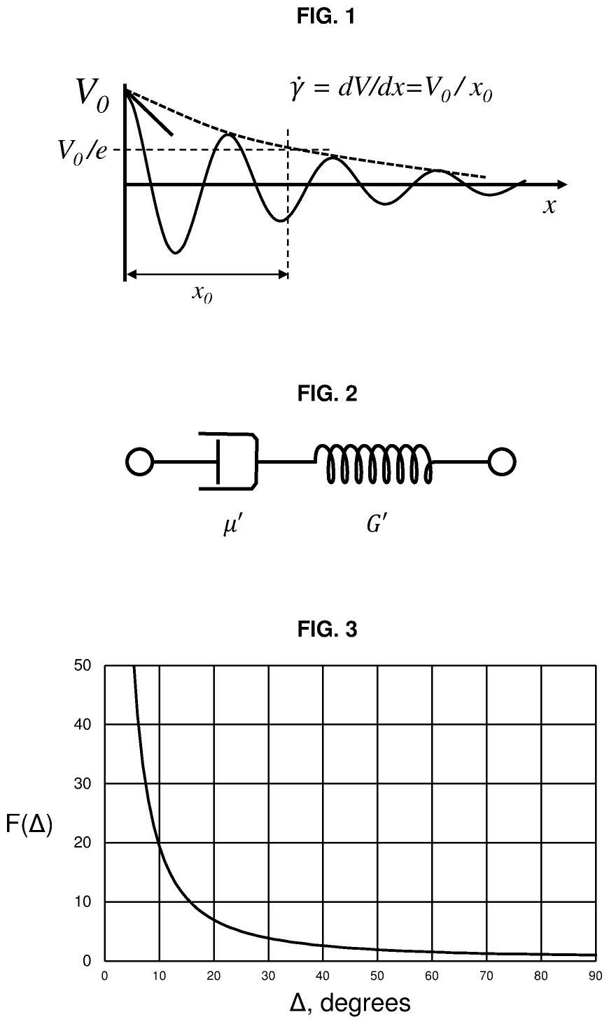

FIG. 1 illustrates a shear wave generated from an oscillating surface in a fluid, shown as a graph of velocity, V, against distance x from the surface. The amplitude of the fluid velocity decays with increasing distance x from the surface. The velocity at the surface is Vo.

The degree of change in velocity over distance x gives the velocity gradient, also known as the shear rate:

$$\dot{γ} = \frac{dV}{dx} \qquad Equation 1$$

The velocity gradient or shear rate at the oscillating surface are important in determining the damping force arising at the oscillating surface via the shear stress, τ, given by:

$$τ = μ \dot{γ} \qquad Equation 2$$

The shear stress and the oscillatory displacement of the surface give rise to ‘work done’, leading to the dissipation of energy. Since the Q factor may be understood to represent a ratio of energy stored to energy lost in oscillation of a resonator, an increase in shear stress leads to an increase in dissipation of energy, which leads to a decrease in Q factor and an increase in its inverse, the loss factor. A measured loss factor or Q factor is therefore an indication of a degree of viscosity. But it also depends on the shear rate at the surface.

The shear wave propagation depth is the distance over which the amplitude of the shear wave drops to a factor of 1/e of its starting amplitude (where e is the base of natural logarithms and 1/e is approximately 0.37). This value is sometimes referred to as the ‘penetration depth’ or ‘skin depth’.

The shear rate at the surface varies inversely with propagation depth.

$$\dot{γ} = - \frac{{V}_{0}}{{x}_{0}} \qquad Equation 3$$

The propagation depth, x₀, may be written as:

$${x}_{0} = \frac{1}{sin Δ / 2 \sqrt{2 sin Δ \frac{ω ρ}{{μ}^{′}}}} \qquad Equation 4$$

where ω is the angular frequency, ρ is the fluid density, and Δ is the angle of the loss tangent, also known as the loss angle, and is dependent on ω, μ′, and G′.

The propagation depth is highly dependent on viscosity and elasticity through the loss angle Δ. The propagation depth also depends on frequency and density, although these are relatively 5 invariable.

A high degree of elasticity unfavourably skews the shear rate. Increasing elasticity extends the propagation depth and therefore lowers the shear rate. This alters the loss factor (or Q factor), from which viscosity is inferred. This means that significant errors in measuring viscosity may arise, particularly in highly non-Newtonian fluids.

FIG. 2 illustrates a simple model for a viscoelastic fluid as a spring and damper system. The apparent viscosity, μ*, has units of Pa·s and is dependent on the dynamic viscosity, μ′, (units of Pa·s) the storage modulus, G′, which represents the elasticity (units of Pa), and the angular frequency, ω, (units of s⁻¹), and is given by the expression:

$${μ}^{*} = \sqrt{{( {μ}^{′} )}^{2} + {( \frac{{G}^{′}}{ω} )}^{2}} \qquad Equation 5$$

The loss modulus, G″, has units of Pa and is the product of the dynamic viscosity, μ′, and the angular frequency, ω.

The loss tangent is given by:

$$tan Δ = \frac{{G}^{″}}{{G}^{′}} = \frac{{μ}^{′} ω}{{G}^{′}} \qquad Equation 6$$

The techniques of this disclosure exploit the dependency of shear wave propagation depth on fluid viscosity and elasticity. The two surfaces are separated by a gap that allows the transition of a wave between a primary oscillating surface, used as a detector, and a secondary oscillating surface, used as an irradiator. The primary surface is irradiated by the secondary surface. The surfaces can be connected (e.g. rigidly) to the same oscillator/resonator or can be independent oscillators/resonators.

The effective or prevailing shear rate at the detector surface is modified by the wave emanating from the irradiating surface. The degree to which the shear rate is modified is dependent on the irradiation intensity and the relative phase between the incident wave in the vicinity of the detector and a shear wave emanating from the detector.

The irradiation intensity, or amplitude of the irradiating shear wave at the detector, varies based on the propagation depth and the distance between the primary and secondary surfaces. The phase of the irradiating shear wave at the detector depends on the wavelength of the shear wave within the fluid and the distance between the primary and secondary surfaces. Both irradiation intensity and relative phase vary with the dynamic viscosity u′ and with the storage modulus G′. The combination of shear waves emanating from the detector with irradiating shear waves at the detector can lead to interference, whether constructive or destructive.

A net destructive interference at the detector arises when the relative phase between the shear waves at the detector is such that instantaneous velocities of the shear waves have opposite signs, leading to an effective shear rate that is less than would be experienced in the absence of the irradiating shear wave (for example, more than 5% lower, more than 10% lower, more than 20% lower, more than 30% lower, more than 40% lower, or more than 50% lower).

A net constructive interference at the detector arises when the relative phase between the shear waves at the detector is such that instantaneous velocities of the shear waves have the same sign, leading to an effective shear rate that is greater than would be experienced in the absence of the irradiating shear wave (for example, more than 5% greater, more than 10% greater, more than 20% greater, more than 30% greater, more than 40% greater, or more than 50% greater).

At the detector, the shear waves emanating from the detector at the detector may be assumed to have the same velocity as the detector itself.

In some configurations, each of a pair of oscillating surfaces may have the potential to be both a detector and an irradiator to each other. Therefore a net constructive or destructive interference from the combination of shear waves may arise at either or both of the oscillating surfaces.

The shear rate of the fluid at the detector resulting from the interference affects the Q factor (or loss factor). Destructive interference at the detector leads to a lower Q factor and therefore a higher loss factor. Constructive interference at the detector leads to a higher Q factor and therefore a lower loss factor. This variation in Q factor (or loss factor) is therefore related to fluid viscosity (via μ′) and elasticity (via G′).

An element underpinning the techniques of this disclosure is the dependency of the shear wave propagation depth on the elasticity of the fluid. This is illustrated by rewriting Equation 4 as a product of a viscous-only skin depth and an elastic component:

$${x}_{0} = \sqrt{\frac{2 {μ}^{′}}{ωρ}} \cdot ( \frac{1}{sin \frac{Δ}{2}} \cdot \sqrt{\frac{1}{2 sin Δ}} ) = \sqrt{\frac{2 {μ}^{′}}{ω p}} \cdot F ( Δ ) \qquad Equation 7$$

where tan Δ=ωμ′/G′ and F(Δ) is the quantity within parentheses, which is the elastic component.

FIG. 3 is a graph of F(Δ) plotted against Δ (in units of degrees), wherein F(Δ) is the elastic component of x₀ in Equation 7 and equal to 1/(sin(Δ/2)·\sqrt{2·sin Δ}).

The quantity F(Δ) is plotted because it represents the effect of elasticity on the propagation depth. This quantity is 1 for Δ equal to 90°, i.e. a purely viscous fluid where tan Δ→∞. For a mildly viscoelastic fluid, for which 1<tan Δ<∞ (i.e. for values of A in the range 45°<Δ<90°, the plotted quantity F(Δ) remains approximately 1. This means that the shear wave propagation depth for mildly viscoelastic fluids might reasonably be approximated as the viscous-only shear wave propagation depth. But the plotted quantity increases at an increasing rate as Δ decreases. As Δ decreases, the viscous-only shear wave propagation depth becomes an increasingly inaccurate approximation of the shear wave propagation depth. As Δ approaches 0, the plotted quantity F(Δ) would approach infinity. Therefore, for more strongly viscoelastic fluids, such as fluids for which tan Δ<1, the effect of viscoelasticity on the shear wave propagation depth is more important and it may become more important to take into account the effect of viscoelasticity when making measurements of fluid properties.

It is noted from Equation 7 that, for fluids with a relatively low value of μ′ and a relatively low value of G′, the shear wave penetration depth is relatively low compared to fluids with a relatively high value of μ′ and a relatively high value of G′.

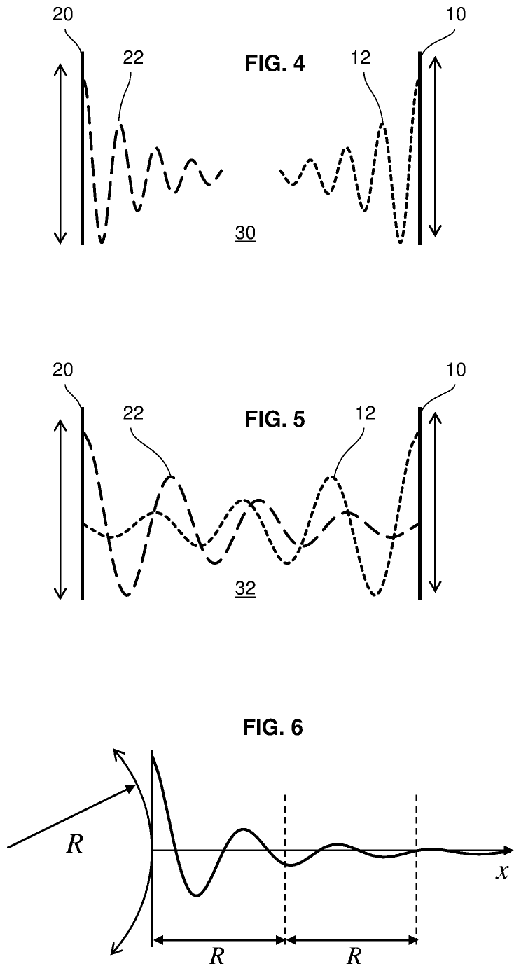

FIG. 4 illustrates shear wave velocity fields generated by a primary oscillating surface 10 (i.e. the detector) and a secondary oscillating surface 20 (i.e. the irradiator), the surfaces oscillating in synchronized fashion in phase with each other, the surfaces separated by a gap distance filled with a first viscoelastic fluid 30 that has relatively low μ′ and relatively low G′. The primary oscillating surface 10 generates a shear wave 12 that propagates a distance into the first viscoelastic fluid 30. The secondary oscillating surface 22 generates a shear wave 22 that also propagates a distance into the first viscoelastic fluid 30. The shear waves 12, 22 do not interfere with each other because the relatively low μ′ and relatively low G′ mean that the shear waves 12, 22 do not cross the gap between the primary oscillating surface 10 and the secondary oscillating surface 20; they decay without giving rise to any interference.

FIG. 5 illustrates shear wave velocity fields generated by the same primary oscillating surface 10 and secondary oscillating surface 20 separated by the same gap as in FIG. 4, the primary and secondary oscillating surfaces oscillating together in the same synchronized fashion as shown in FIG. 4. But in FIG. 5, the viscoelastic fluid 32 that fills the gap has relatively high μ′ and relatively high G′. The relatively high μ′ and relatively high G′ mean that the shear waves 12, 22 do extend across the gap between the primary oscillating surface 10 and the secondary oscillating surface 20. The shear waves 12, 22 can interfere with each other at the primary and secondary oscillating surfaces. This means that the effective shear rate at the primary oscillating surface 10 (i.e. the detector) is affected by the shear wave 22 generated at the secondary oscillating surface 20 (i.e. the irradiator).

It is not required that both μ′ and G′ be high in order for a propagation depth to be long. For example, a long propagation depth may be achieved with high μ′ or G′ alone.

For a given range of μ′ and G′, parameters of the arrangements shown in FIGS. 4 and 5 can be selected to affect the shear rate at the detector surface via interference. Such parameters include one or more of the following: i) gap distance, ii) frequency, iii) surface radius, iv) surface shape.

Returning to FIG. 1, the travelling plane wave velocity may be expressed as:

$$v ( x , t ) = {V}_{0} {e}^{- α x} {e}^{i ( ω t - βx )} \qquad Equation 8$$

where α is the attenuation factor, equal to 1/x₀, and β is the wavelength factor, equal to 2π/λ, where λ is the wavelength.

For a shear wave in the viscoelastic fluid, a and B are given by the following expressions:

$$α = sin ( Δ / 2 ) \sqrt{\frac{sin ( Δ ) ω ρ}{{μ}^{′}}} \qquad Equation 9$$

$$β = cos ( Δ / 2 ) \sqrt{\frac{sin ( Δ ) ω ρ}{{μ}^{′}}} \qquad Equation 10$$

The amplitude of the velocity oscillations decreases with distance from the originating surface by α=1/x₀. The phase of the velocity oscillations changes with distance from the originating surface according to the wavelength factor, β. Both α and β are fluid-dependent and determine the degree of interference of velocity fields and therefore the degree to which the effective shear rate at a detecting surface is modified.

The discussion thus far has assumed a flat oscillating surface that oscillates in-plane to generate the shear waves. But shear waves may be generated from non-flat surfaces. For example, shear waves may be generated from concave or convex surfaces undergoing torsional vibration.

FIG. 6 illustrates the propagation of a shear wave from a surface having a radius of curvature R undergoing torsional vibration.

For a convex surface, the travelling wave velocity may be expressed as:

$$v ( x , t ) = {V}_{0} \vert \frac{R}{R + x} \vert {e}^{- α x} {e}^{i ( ω t - β x )} \qquad Equation 11$$

In this equation, the phase of the wave varies as before with distance x according to the wavelength factor β. But the amplitude of the wave varies differently. The amplitude is not just affected by α, the attenuation factor, but also due to geometric considerations as the wave's energy is spread over an increasingly large region with increasing distance from the originating surface. Therefore, the amplitude varies according to (R/(R+x))e^{-αx}.

For a concave surface, the travelling wave velocity may be expressed as:

$$v ( x , t ) = {V}_{0} \vert \frac{R}{R - x} \vert {e}^{- α x} {e}^{i ( ω t - β x )} \qquad Equation 12$$

As with the convex surface, the phase variation of the wave with distance depends on β. The amplitude is affected by α and by a geometric term, although the geometric term is different in the case of the concave surface. The geometric term accounts for the focusing of the wave's energy in an increasingly small region with increasing distance from the originating surface. Therefore, the amplitude varies according to |R/(R−x)|e^{-αx}.

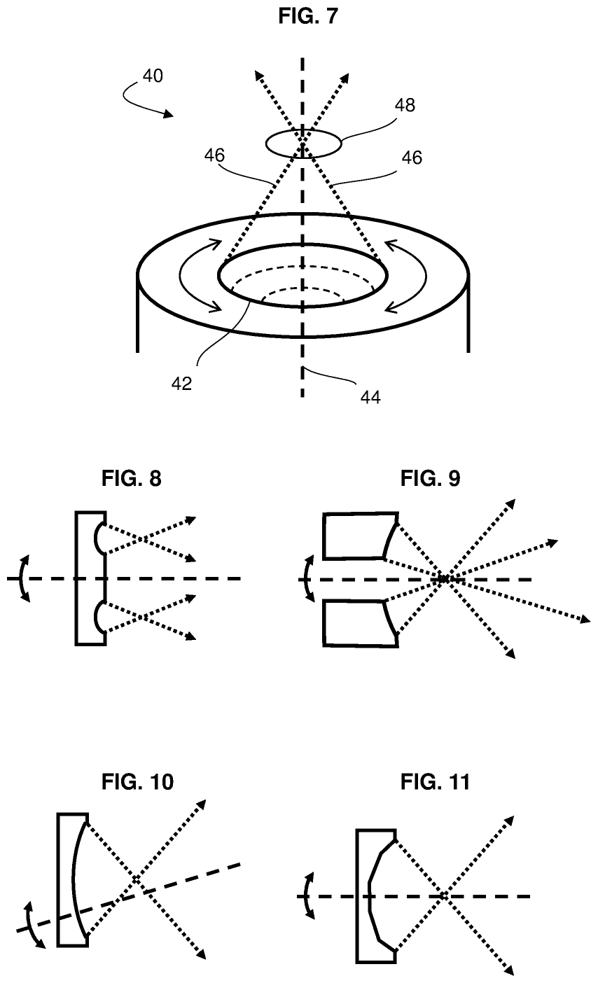

FIG. 7 shows an arrangement in which a vibratory transducer 40 is provided with a concave surface 42, the concave surface 42 comprising a spherical recess in a surface of the vibratory transducer. The vibratory transducer 40 is vibrated torsionally about an axis of rotation 44. The axis of rotation 44 extends through the centre of the concave surface 42. While the vibratory transducer torsionally vibrates about the axis of rotation 44, shear wave ‘rays’ 46 extend from all locations on the concave surface 42, where the shear wave transverse velocity of each ray 46 at its respective origin location on the concave surface 42 is aligned with the velocity of the origin location on the concave surface 42 due to the no slip condition. Due to the spherical profile of the concave surface, which means that there is a constant radius of curvature, all rays 46 focus to single location defined by the radius of curvature and a surface normal direction from the concave surface 42.

The particular angular location around the axis of vibration 44 of an origin point of a ray 46 defines the direction of velocity of the concave surface 42 at that origin point. For any two points on the concave surface 42 that have the same longitudinal position along the axis of rotation 44, the surface velocities will be equal in magnitude but with a direction and phase that depends on the angular offset of the two points about the axis of rotation 44.

If two origin points of rays 46 are offset from each other on the concave surface 42 about the axis of rotation 44 by 180°, then the phases of the shear waves of the rays 46 will be offset from each other by 180°.

In these conditions, the travelling wave velocity is given by the following expression:

$$v ( x , t ) = {V}_{0} ( \frac{R}{R - x} ) {e}^{- α x} {e}^{i ( ω t - β x )} \qquad Equation 13$$

In these conditions, the concave surface not only provides a focusing effect but also an amplitude inversion effect due to the geometric term (R/(R−x)), the amplitude inversion depending on whether the distance x is greater than or less than the radius of curvature, which determines the sign of this geometric term.

The example shown in FIG. 7 represents a simple geometry of a recess with the profile of a constant-curvature sphere that is symmetric about the axis of rotation. It ignores any spreading effects of the generated shear waves. A practical recess might not be perfectly smooth, or the radius of curvature might not be constant, or the recess might not be symmetric about the axis of rotation. In practice, the focus of the shear waves will therefore not be a single point in space but will be a focal region. But if the recess is concave and is rotated in a fluid about an axis in the fluid that extends through and outward from the surface of the recess such that portions of the surface recess spaced from the axis of rotation on opposite sides of the axis of rotation direct waves toward the axis of rotation, then a focal region will be formed, behind which an amplitude inversion may occur.

Therefore a net destructive interference arises when, at the detector, shear waves from the irradiator and detector have opposite signs, regardless of whether it is achieved through the phase difference due to the distance between detector and irradiator or through an amplitude inversion due to a focusing effect. At the detector, the shear waves emanating from the detector at the detector may be assumed to have the same velocity as the detector itself.

FIG. 8 illustrates a configuration in which a smooth concave recess extends in a ring around the axis of rotation (shown in cross section). This configuration would not be expected to lead to amplitude inversion within or behind the focus region because the ‘rays’ that focus together are all in phase with each other. At each point within or behind the focus, all of the rays come from the same angular position about the axis of rotation.

FIG. 9 illustrates a configuration in which a smooth concave recess rotates angularly about an axis of rotation. The concave recess has constant curvature but does not provide a complete spherical surface. In particular, there is no curved surface generating shear waves in the direction of the axis of rotation and aligned with the axis of rotation. An amplitude inversion will be expected to occur only in the region where the shear waves from either side of the axis of rotation superimpose, which corresponds to a phase inversion. Away from this region, there will be no amplitude inversion along the axis because of the absence of shear waves in the direction of the axis. There will also be no amplitude inversion in the shear wave paths behind the focus because all of the shear waves in such regions are in phase.

FIG. 10 illustrates a configuration in which a smooth concave recess of spherical shape is rotated about an axis in the fluid. The axis extends through the concave recess, but the axis of rotation is not aligned with the centre of the concave recess. It does not pass through either the axial centre of the concave recess or the resulting focal point. Rotation about this axis will not cause focusing of shear waves to the same extent as shown in FIG. 7 because the wave amplitudes either side of the axis of rotation will be mismatched. But there will be expected to be some degree of amplitude inversion behind the focus. Amplitude matching would be improved by moving the axis of rotation closer to the centre of the recess and aligning it to extend through the focus.

FIG. 11 illustrates a configuration in which the recess is formed of multiple conical portions. A lateral cross section through the axis of rotation, about which the recess is axially symmetric, shows the profile of the recess to be piecewise linear. This configuration will provide a degree of focusing but it will not focus as well as a continuous smooth profile such as the configuration of FIG. 7. But it will still be expected to give rise to an amplitude inversion at or behind the focus to at least some degree.

In general, to achieve an amplitude inversion behind a focus of a recess, the concave region will be symmetric about the axis of rotation. For efficient focusing, the concave recess will preferably be smooth in lateral cross section. Preferably the concave recess will be spherical, i.e. have a constant radius of curvature (when viewed in lateral cross section) such that all generated shear waves focus to a single point. In some embodiments the concave recess may have other cross section profiles such as elliptic or parabolic according to application requirements.

An amplitude inversion combined with focusing as shown in these examples provides allows for a detector surface, such as an elongate member or plane surface, to be located in the region of amplitude inversion. If the elongate member is rotating with the shear-wave generating surface in the recess, then shear waves emanating from the detector surface interfere destructively with shear waves emanating from the recess. The net destructive interference gain is high because the focusing of the shear waves amplifies the interfering wave and the detector is in located in the interference focal zone.

Without the amplitude inversion, it would be necessary to locate the detector surface at a distance from the generating surface that is governed by β in order to experience destructive interference with shear waves emanating from the generating surface, and β varies according to the properties of the fluid: Δ, ρ, and μ′, as well as the frequency of vibration w.

But using a concave shear wave generating surface in which out-of-phase shear waves are generated on either side of the axis of rotation, an out-of-phase region can be created in which destructive interference can occur that does not depend on β, and so advantageously does not depend on μ′ or Δ.

Returning to the arrangement shown in FIG. 5, we consider the effect of the interference of the shear waves 12, 22 on the apparent shear rate at the detector surface.

The velocity, at a location in the gap, of the shear wave 22 originating from the secondary oscillating surface 20 may be expressed as:

$${v}_{s} = {A}_{s} cos ( {ω}_{s} t - {ϕ}_{S} ) \qquad Equation 14$$

where Aₛ represents the amplitude-varying term of the shear wave 22, ωₛ represents the frequency of oscillation at the secondary oscillating surface 20, and ϕₛ represents the phase of the shear wave 22 at that location.

The velocity of the shear wave 12 originating from the primary oscillating surface 20 at that same location in the gap may be expressed as:

$${v}_{p} = {A}_{p} cos ( {ω}_{p} t - {ϕ}_{p} ) \qquad Equation 15$$

where Aₚ represents the amplitude-varying term of the shear wave 12, ωₚ represents the frequency of oscillation at the primary oscillating surface 10, and ϕₚ represents the phase of the shear wave 12 at that location.

The linear combination of the shear waves 10, 12 at that location is given by:

$${v}_{p} + {v}_{s} = {A}_{p} cos ( {ω}_{p} t - {ϕ}_{p} ) + {A}_{s} cos ( {ω}_{s} t - {ϕ}_{s} ) \qquad Equation 16$$

The shear rate at that location is given by:

$$\dot{γ} = \frac{d}{d x} ( {v}_{p} + {v}_{s} ) \qquad Equation 17$$

Depending on the relative amplitude and phase of the two waves, either destructive interference (leading to decreasing Q factor) or constructive interference (leading to increasing Q factor) may be experienced.

The amplitude is determined by the fluid attenuation factor a and the surface radius of curvature. The phase is determined by the wavelength factor β and the propagation distance. As set out in Equation 9 and Equation 10, both α and β depend on fluid properties.

The inventors have recognised that an appropriately designed system can provide a measure of Q factor (or its inverse, the loss factor) that varies monotonically with increasing μ′ and G′. This is preferably achieved by ensuring that phase and amplitude conspire to create ever increasing destructive interference against increasing μ′ and G′, yielding a proportionally increasing loss over the range.

As discussed above, a measured loss factor or Q factor is an indication of a degree of viscosity but it also depends on the shear rate at the surface. The shear rate is affected by other factors including the elasticity of the fluid, leading to increasingly non-linear and even non-monotonic relationships between loss factor or Q factor and viscosity. The techniques of this disclosure may employ destructive interference that increases with μ′ and G′ to provide a monotonic relationship between Q factor or loss factor and a measure of μ′ or G′, or any other material property that may be obtained from a measure of μ′ or G′.

Ideally, for a measurement device, the output signal varies linearly and monotonically with the measured variable over a target range. If the output signal does not vary linearly but does vary monotonically over the target range, then such output signal may still be converted directly to an estimate of the measured variable via numerical approximation, such as via curve fitting techniques such as nonlinear regression using nonlinear functions such as nonlinear polynomials. If the output signal is not monotonic with the measured variable over a target range, then determining an estimate of the measured variable becomes more difficult. For example, if multiple values of μ′ would give rise to the same output signal (e.g. loss factor or Q factor) then that value of output signal does not conclusively indicate μ′. Therefore a monotonic relationship between the measured variable and the output signal (e.g. loss factor or Q factor) is advantageous.

In the techniques of this disclosure, gap dimensions and vibration frequency are selected for given fluid properties so that variations in wave factors a and B alter the loss factor or Q factor monotonically with μ′ and G′.

Before particular embodiments according to the techniques of this disclosure are described directly, it will be helpful to set out some general points on the techniques of this disclosure, wherein embodiments may adhere to one or more of the following points in any combination:
Architectures, including phase and amplitude supporting architectures, can be set up for one or both of destructive and constructive interference.
The promotion of destructive interference may be advantageous for achieving more helpful monotonic behaviour of the measured Q factor or loss factor for varying material properties.
Each of a pair of oscillating surfaces may have the potential to be both a detector and an irradiator to each other.
A simple architecture may have both primary and secondary oscillating surfaces as part of the same resonator or oscillating body, although the primary and secondary oscillating surfaces are not required to be so arranged.
Resonator surfaces can be designed to have zero relative phase in their oscillations or to have a defined phase offset.
Shear waves originating from convex irradiators may have higher amplitude loss over a distance than flat or concave irradiators due to geometric considerations of the spreading waves (i.e. radial or geometric damping as discussed below).
Shear waves originating from concave irradiators may be focused at a distance in accordance with the radius of curvature of the irradiator surface, leading to increased amplitude at the focus.
In some configurations, out-of-phase shear waves from opposing sides of a convex irradiator may destructively interfere leading to an amplitude inversion in the shear wave field behind the focus.
Oscillating pin or ring monopole detectors concentrate their velocity field over a short distance due to radial (geometric) damping, which may allow their effective placement at the focus of a convex irradiator.
Determining the loss factor may be more useful in practice than determining the Q factor (its inverse).
Wave properties and gap distance (surface separation) may be selected so that the measured loss factor increases monotonically with increasing μ′ and G′.
The frequency of operation is arbitrary, but many practical implementations will use frequencies in the region of 100 Hz to 100 kHz, preferably 200 Hz to 10 kHz, more preferably 300 Hz to 5 kHz, and more preferably 500 Hz to 2 kHz, such as frequencies at or around (e.g. +/−10%) 1 kHz.
While Q factor (or loss factor) may be used to measure the shear stress, the techniques of this disclosure are not so limited and may include as an alternative the measurement of shear stress via direct torque measurement. The use of constructive and destructive interference as described herein may provide the same benefits regardless of whether Q factor or loss factor are measured.
The techniques of this disclosure are presented in the context of shear waves, but the same principles apply to oscillations from surfaces generating pressure waves (acoustic waves or P waves) that constructively or destructively interfere. It is noted however that such waves have longer wavelengths than shear waves and so oscillations at considerably higher frequencies be required, or otherwise the system dimensions will need to be increased to account for the longer wavelengths.
The techniques of this disclosure may advantageously be implemented using torsional vibratory devices to produce shear waves. However, the oscillating surfaces may alternatively be provided by surfaces of mechanical oscillators vibrating in-plane, i.e. in a lateral or longitudinal vibrational modes, in addition to or instead of a torsional mode.

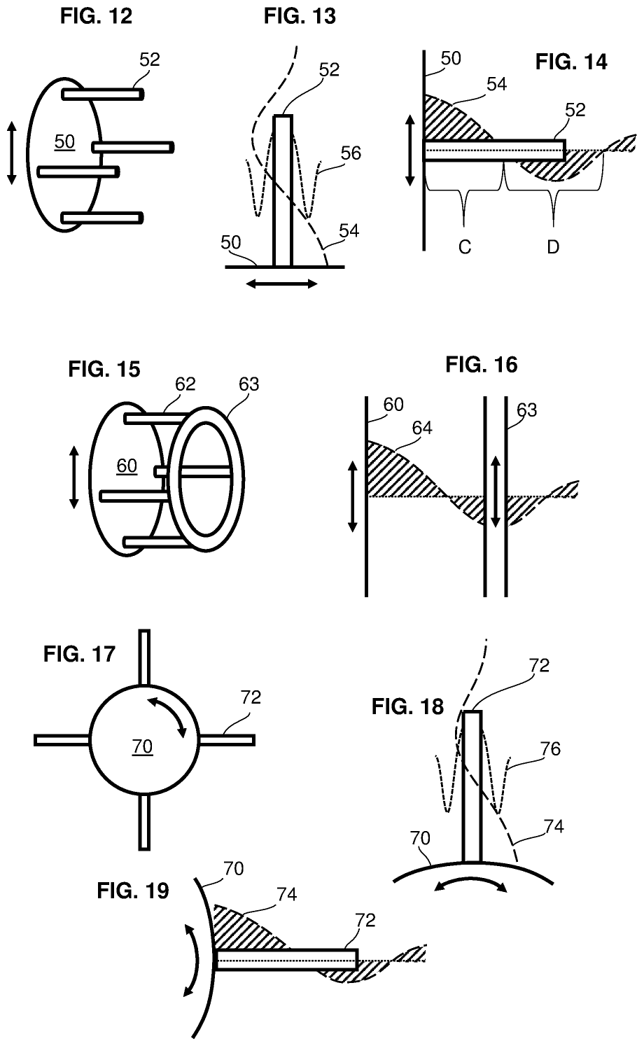

FIG. 12 illustrates a configuration for a vibratory transducer in accordance with the techniques of this disclosure in which a plurality (four in this case) of elongate members 52, such as pins, extend perpendicularly outward from a flat disc base 50. The flat disc base 50 is configured to vibrate in plane, i.e. planar in a direction perpendicular to the normal of the plane or torsional vibration about an axis parallel to the normal of the plane. Optionally, the elongate members may be elongate members as described below and in GB application no. 2207881.0 filed 27 May 2022, which is hereby incorporated by reference into the present disclosure; such elongate members are characterised by a width, a half width that is equal to half of the width, and a length that is greater than the width, wherein the half width is less than a propagation depth of a shear wave in the fluid at the vibration frequency, more preferably less than 50% of the propagation depth. The propagation depth of the shear wave may be 1/α, wherein a is given by Equation 9. During vibration, the flow of fluid around the elongate member may be laminar flow.

In this configuration, the flat disc base 50 may be understood as an irradiator and an elongate member 52 may be understood as a detector.

In a configuration, the flat disc base 50 may be configured to vibrate torsionally about a longitudinal axis of the base instead of laterally.

FIG. 13 illustrates the shear wave 54 generated by the flat base 50 and the shear waves 56 generated by the elongate member 52. Geometric damping as described below may cause the shear waves 56 generated by the elongate member to take the form shown in FIG. 8, wherein the shear wave amplitude rapidly decays due to geometric considerations. On the assumption that the elongate member 52 is sufficiently rigid that any flexural vibrate of the elongate member 52 at the frequencies of operation is negligible and can be ignored, the elongate member 52 vibrates in phase with the flat base 50. The shear wave 54 generated by the flat base 50 varies in value with increasing distance from the flat base 50 in accordance with B.

FIG. 14 further illustrates the variation in phase of the shear wave 54 generated by the flat base 50 along the length of the elongate member 52. In a first range, labelled C in FIG. 9, the shear wave 54 generated by the flat base 50 is in phase with the motion of the elongate member 52 and therefore in phase with the any shear wave 56 generated from that portion of the elongate member 52. In this region, constructive interference occurs between the shear wave 54 generated by the flat base 50 and the shear wave 56 generated by the elongate member 52. In a second range, labelled D in FIG. 9, the shear wave 54 generated by the flat base 50 is out of phase with the motion of the elongate member 52 and therefore out of phase with the any shear wave 56 generated from that portion of the elongate member 52. In this region, destructive interference occurs between the shear wave 54 generated by the flat base 50 and the shear wave 56 generated by the elongate member 52. As discussed above, destructive interference may be preferred in practice. The arrangement shown in FIG. 9 may provide only a modest destructive interference gain due to the constructive interference in the region C adjacent the base of the elongate member 52 where it contacts the flat base 50.

FIG. 15 illustrates a configuration for a vibratory transducer in accordance with the techniques of this disclosure in which in which a plurality (four in this case) elongate members 62, such as pins, extend perpendicularly outward from a flat disc base 60. In addition, the distal ends of the elongate members 62 attach to a ring member 63. The ring member 63 encircles an axis that is perpendicular or approximately perpendicular to the flat base 60. Each of the elongate members 63 has the same or a similar length such that, around the ring member 63, each portion is of the ring member 63 is approximately the same distance from the flat base 60. Optionally both the ring member 63 and elongate members 62 may optionally have sufficiently narrow widths to provide geometric damping as described below. The flat disc base 60 is configured to vibrate in plane, i.e. planar in a direction perpendicular to the normal of the plane or torsional vibration about an axis parallel to the normal of the plane.

In this configuration, the flat disc base 60 may be understood as an irradiator and an elongate member 62 may be understood as a detector and also the ring member 63 may be understood as a detector.

The shear waves generated by motion of the elongate members 62 interact with shear waves 64 generated by the motion of the flat disc base 60 in the same manner illustrated in FIGS. 7 to 9. But this configuration may provide greater amount of net destructive interference, i.e. the effective interference taking into account constructive interference contributions and destructive interference contributions.

In a configuration, the flat disc base 60 may be configured to vibrate torsionally about a longitudinal axis of the base instead of laterally.

FIG. 16 illustrates the ring member 63 in the shear wave 64 generated by motion of the flat base 60. The ring member 63 is connected to the elongate members 63, which are assumed to be rigid such that the elongate member 63 moves in phase with the flat base 60 and the ring member 63 moves in phase with the flat base 60. The whole of the ring member 63 is located at a distance from the flat base 60 such that the shear wave 64 generated by the motion of the flat base is out of phase with the ring member 63 at the location of the ring member 63 and therefore out of phase with any shear wave generated from the motion of the ring member 63. Therefore destructive interference occurs between the shear wave 64 generated by the flat base 60 and any shear wave generated by the ring member 63. Therefore while the elongate members 62 may provide only a modest amount of net destructive interference, the addition of the ring member 63 provides an improved amount of net destructive interference when the ring member 63 located at a position where shear waves generated by motion of the flat base 60 are out of phase with the flat base 60, and therefore out of phase with the ring member 63.

FIG. 17 illustrates a configuration for a vibratory transducer in accordance with the techniques of this disclosure in which a plurality (four in this case) elongate members 72, such as pins, extend radially outward from a shaft 70 that is circular in cross section, and is configured to vibrate torsionally about a longitudinal axis of the shaft 70. Optionally, the elongate members 72 may optionally have sufficiently narrow widths to provide geometric damping as described below.

In this configuration, the radially facing cylindrical outer surface of the shaft 70 may be understood as an irradiator and an elongate member 72 may be understood as a detector.

FIG. 18 illustrates the different shear waves generated by motion of elements of this configuration. The torsional vibration of the shaft generates shear waves 74 extending radially outward from the cylindrical surface of the shaft 70. As discussed above in relation to Equation 11, the amplitude of the shear waves 74 with increasing distance from the shaft 70 is not just affected by the attenuation factor but also by geometric considerations. The elongate members 72 move in phase with the shaft 70 and generate shear waves 76 from their motion in the fluid. At some locations along the length of the elongate member 72, the motion of the elongate member is in phase with the amplitude of the shear wave 74 generated from the shaft 70. At other locations along the length of the elongate member 72 the motion of the elongate member is out of phase with the amplitude of the shear wave 74 generated from the shaft 70.

FIG. 19 illustrates the locations in which the amplitude of the shear wave 74 generated from the cylindrical surface of the shaft 70 and that extend radially outward from the shaft 70 is in phase with the motion of the shaft 70, and therefore the motion of the elongate member 72. It can be seen that, in a region closest to the base of the elongate member 72 at which the elongate member 72 connects to the shaft 70, the amplitude of the shear wave 74 is in phase with the motion of the elongate member 72, and therefore in phase with a shear wave generated by the motion of the elongate member 72, leading to constructive interference at this portion of the elongate member 72. In a next adjacent region that is further from the shaft 70, the amplitude of the shear wave 74 is out of phase with the motion of the elongate member 72, and therefore out of phase with a shear wave generated by the motion of the elongate member 72, leading to destructive interference at this portion of the elongate member 72. The net destructive interference gain is modest due to the constructive region at the base of the elongate member 72. Compared with the arrangements shown in FIGS. 12 to 18, the irradiating surface is convex, leading to additional attenuation with increased radial distance due to geometric considerations.

FIG. 20 illustrates a configuration for a vibratory transducer in accordance with the techniques of this disclosure that is based on that shown in FIGS. 17 to 19, but in which the distal ends of the elongate members 73 are connected to a ring member 73. The ring member 73 may optionally have a sufficiently narrow width to provide geometric damping.

FIG. 21 illustrates the location of the ring member 73 in a region in which the shear waves 74 generated by the motion of the shaft 70 are out of phase with the motion of the shaft 70 and therefore out of phase with the motion of the ring member 73. Shear waves generated by the motion of the ring member 73 are out of phase with the shear wave 74 generated by the motion of the shaft 70 at this distance radially outward from the shaft 70, leading to destructive interference. While the irradiating outer surface of the shaft 70 is convex, and therefore the shear waves 74 undergo additional geometric attenuation with radial distance, the net destructive interference gain may be high due to the ring member 73 detector being located in the destructive interference region only as shown in FIG. 21.

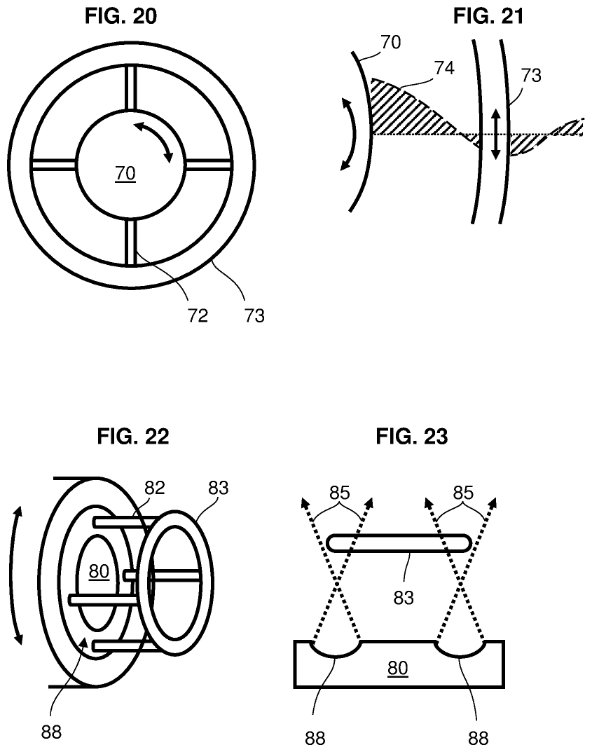

FIG. 22 illustrates a illustrates a configuration for a vibratory transducer in accordance with the techniques of this disclosure that is based on FIG. 15, but where the vibration is torsional and in which shear waves are generated from a concave surface. In particular, the base 80 is in the form of a cylinder configured to vibrate torsionally about its longitudinal axis. At one end of the cylindrical base 80, four elongate members 82 extend outward from the base, the elongate members 82 aligned with the longitudinal axis of the cylindrical base 80. A ring member 83 is connected to the distal ends of the elongate members 82. The end of the base 80 is has a concave groove 88 provided therein, the concave groove 88 extending in a circle around the longitudinal axis of the base 80 that is concentric with the outer cylindrical surface of the base 80. The elongate members 82 are connected to the end of the base 80 within the concave groove 88. In this example, the concave groove has a constant radius of curvature across the groove cross section. However, other examples may have grooves with non-circular cross section profiles, such as elliptical or parabolic cross section profiles. A concave surface or groove in the context of the present disclosure is not required to be smooth however and may encompass surfaces or grooves with piece-wise linear profiles or surfaces of grooves with a mixture of flat and curved portions. An example might be a groove with a V-shaped cross section profile.

FIG. 23 shows a cross section through the end of the base 80 and showing the ring member 83. Elongate members 82 are not shown. Torsional vibration within a fluid of the base 80 about its longitudinal axis causes shear waves to be generated from its end, including from within the concave groove 88. The shear waves emanate in a direction normal to the surface. Shear waves generated from the surface within the concave groove 88 are focused to a region at offset from the base 80, as shown by rays 85 representing the direction of shear wave propagation from the originating surface within the concave groove 88. The circular cross section of the concave groove 88 leads to a shallow region of focus. At distances beyond the region of focus, the resulting amplitude of the shear wave is not expected to undergo an inversion as discussed above in relation to Equation 12. This is because the shear waves that focus together are all in phase.

FIG. 23 illustrates the location of the ring member 83 at a distance from the base 80 beyond the region of focus. Shear waves, shown by rays 85, are not expected to provide an amplitude inversion, relative to the motion of the base 80, beyond the region of focus 89.

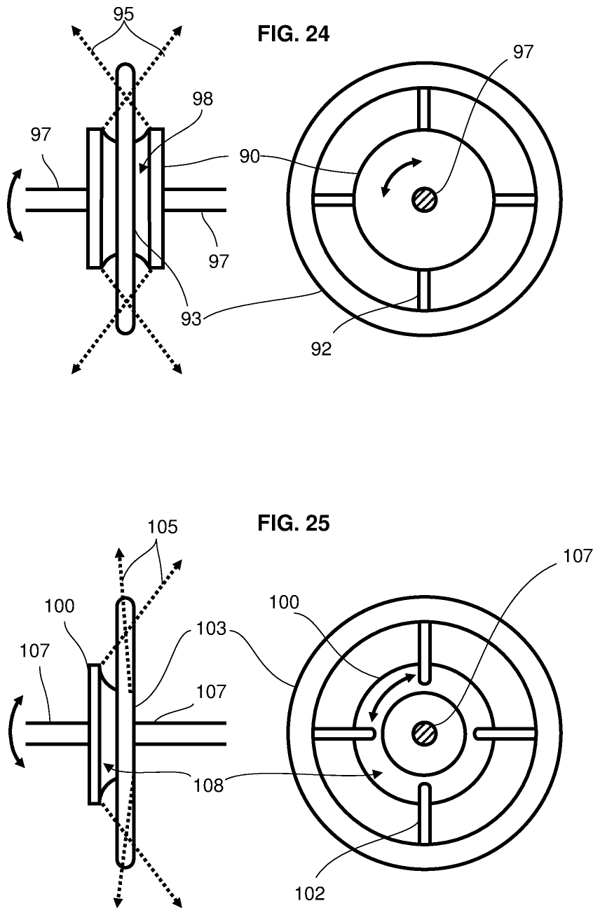

FIG. 24 illustrates an arrangement according to the techniques of this disclosure that can be likened to a ‘pulley wheel’ irradiator and a ring detector. FIG. 24 shows a side view of a fluid-contacting element of a vibratory transducer on the left-hand side and an axially oriented view of the same transducer on the right-hand side. The vibratory transducer comprises a shaft 97 configured to torsionally vibrate about a longitudinal axis. The shaft 97 includes a bob 90 along its length, the bob 90 having a cylindrical form aligned with the axis of the shaft 97. Extending circumferentially around the cylindrical outer surface of the bob 90 there is a concave groove 98 having a circular profile. The bob 90, including concave groove 98, has the form of a pully wheel. Extending radially outward from the concave groove 98 there are a plurality of elongate members 92, the elongate members 92 connected at their distal ends to a ring member 93 that encircles the bob 90 around the concave groove 98. The surface of the concave groove 98 serves as an irradiator for the ring member 93, which serves as a detector.

Rays 95 represent the shear waves emanating from the surface of the concave groove 98. The radius of curvature of the concave groove 98 causes the shear waves emanating from the concave groove 98 to focus at a region in intermediate between the concave groove 98 and the ring member 93.

FIG. 25 illustrates an arrangement according to the techniques of this disclosure that can be likened to a ‘half pulley wheel’ irradiator and a ring detector. FIG. 25 shows a side view of a fluid-contacting element of a vibratory transducer on the left-hand side and an axially oriented view of the same transducer on the right-hand side. The vibratory transducer comprises a shaft 107 configured to torsionally vibrate about a longitudinal axis. The shaft 107 includes a bob 100 along its length, the bob 100 having a cylindrical form aligned with the axis of the shaft 97. While the bob 100 includes a concave groove 108, the concave groove 108 represents a narrowing of the diameter of the bob 100 from a maximum to a minimum value. The concave groove 108 has a circular profile (constant radius of curvature). The bob 100, including concave groove 108, has the form of a half pully wheel. Extending radially outward from the concave groove 108 there are a plurality of elongate members 102, the elongate members 92 connected at their distal ends to a ring member 103 that encircles the bob 100 around the concave groove 108. The surface of the concave groove 108 serves as an irradiator for the ring member 103, which serves as a detector.

Rays 105 represent the shear waves emanating from the surface of the concave groove 108. The radius of curvature of the concave groove 108 causes the shear waves emanating from the concave groove 108 to focus at a region in intermediate between the concave groove 108 and the ring member 103.

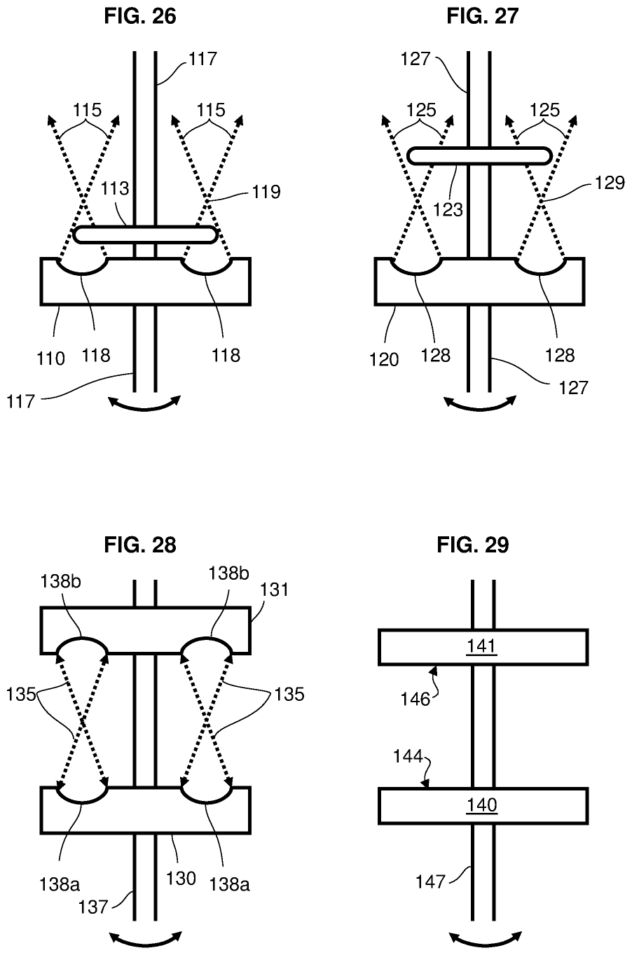

FIG. 26 illustrates an arrangement according to the techniques of this disclosure in which a base or bob 110 on a shaft 117 is provided with a concave groove 118 extending in a circle around the shaft 117. A ring member 113 extends circumferentially around the shaft 117 (connected to the shaft by elongate members not shown) and is located at a position axially offset from bob 110. The shaft 117 and bob 117 vibrate torsionally about the longitudinal axis of the shaft. The ring member 113 is positioned closer to the bob 110 than the region of focus 119, shown with rays 115 emanating from the concave groove 118. This means the amplitude of an interfering shear wave from the concave groove 118 irradiator experienced at the ring member 113 detector is enhanced and interference has a greater effect.

FIG. 27, in accordance with the techniques of this disclosure, illustrates the same arrangement as FIG. 26, except the ring member 123 is located further from the irradiator than the region of focus 129.

FIG. 28, in accordance with the techniques of this disclosure, illustrates an arrangement in which two bobs 130, 131 are each located on a torsionally vibrating shaft 137 at a distance from each other. On a first bob 130, on an axially facing side that faces that second bob 131, there is provided a concave groove 138a extending in a circle around the axially facing side of the first bob 130 around the shaft 137. On the second bob, on an axially facing side that faces that first bob 130, there is provided a concave groove 138b extending in a circle around the axially facing side of the second bob 131 around the shaft 137. Shear waves emanating from the concave groove 138a of the first bob 130 are focused to a region between the two bobs 130, 131 and undergo an inversion beyond the focus region. Shear waves emanating from the concave groove 138b of the second bob 131 are focused to a region between the two bobs 130, 131.

FIG. 29, in accordance with the techniques of this disclosure, illustrates an arrangement in which two opposing discs 140, 141 are mounted on a shaft 147 and configured to vibrate torsionally. A first face 144 of the first disc 140 faces a second face 146 of the second disc 141. Shear waves that propagate across the gap distance between the first face 144 and the second face 146 may interfere with each other constructively or destructively.

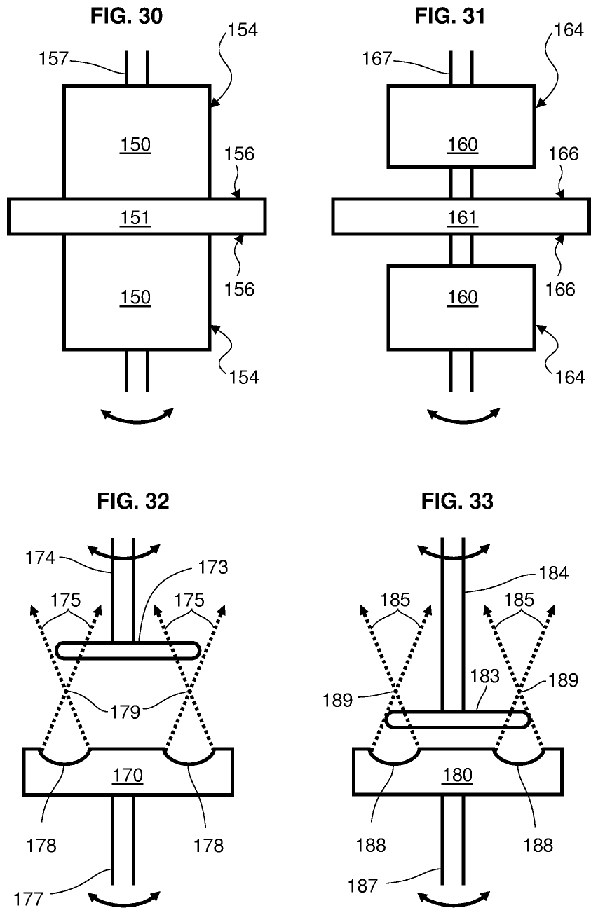

FIG. 30, in accordance with the techniques of this disclosure, illustrates an arrangement in which a bob 150 on a torsionally vibrating shaft 157 is provided with a disc 151 extending circumferentially around the bob 150. The outermost radial extent of the disc 151 is greater than the outermost radial extent of the bob 150. During torsional vibration of the shaft 157, shear waves emanate out radially from the outer cylindrical surface 154 of the bob 150. In addition, shear waves emanate out axially from the axial-facing surfaces 156 of the disc. Such shear waves may interfere with each other constructively or destructively.

FIG. 31, in accordance with the techniques of this disclosure, illustrates an arrangement in which two bobs 160 are located spaced from each other on a torsionally vibrating shaft 167. In between the two bobs 160 there is a disc 161 axially aligned with the bobs 160 and the shaft 167. The outermost radial extent of the disc 11 is greater than the outermost radial extent of either bob 160. During torsional vibration of the shaft 167, shear waves emanate out radially from the outer cylindrical surface 164 of the bob 160. In addition, shear waves emanate out axially from the axial-facing surfaces 166 of the disc. Such shear waves may interfere with each other constructively or destructively. Compared with the arrangement shown in FIG. 30, the arrangement shown in FIG. 31 may offer improved destructive interference. This is because the diameter of the bob 160 detector adjacent to the disc 161 irradiator is much reduced, and so the contributory effect of any constructive interference from this region is much reduced.

FIGS. 32 and 33 illustrate arrangements in accordance with the techniques of this disclosure that are similar to the arrangements shown in FIGS. 26 and 27, except that the ring members 173, 183 are located on a separate shaft 174, 184 from the shaft 177, 187 on which the bob is located. The shafts are configured to vibrate torsionally together to provide the shear wave interference as desired.

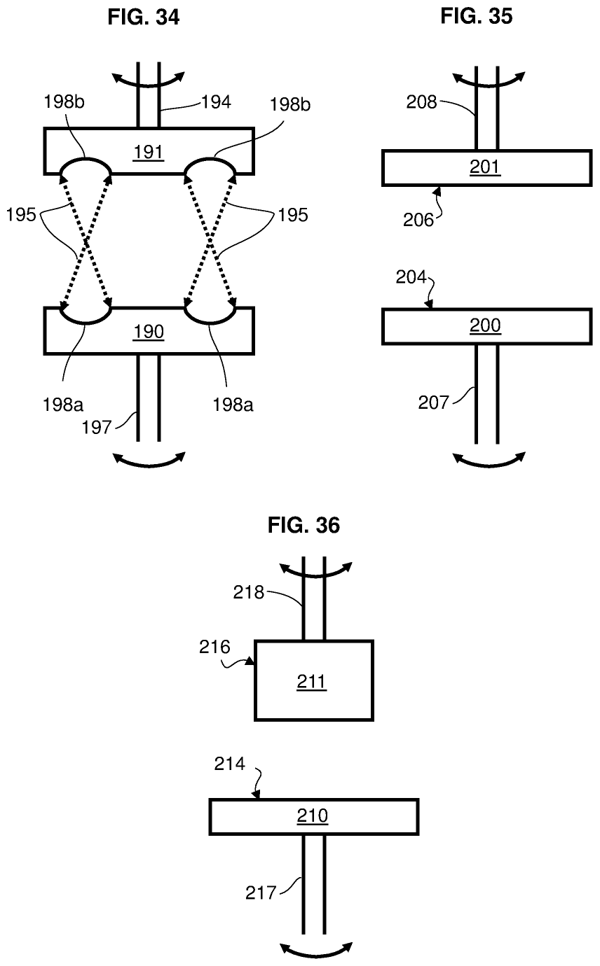

FIG. 34, in accordance with the techniques of this disclosure, illustrates an arrangement in which two bobs 190, 191 are each located on a separate torsionally vibrating shafts 194, 197 at a distance from each other. A first bob 190 torsionally vibrating shaft 137 at a distance from each other. On a first bob 190 attached to shaft 197, on an axially facing side that faces that second bob 191, there is provided a concave groove 198a extending in a circle around the axially facing side of the first bob 190. On the second bob 191 attached to shaft 194, on an axially facing side that faces that first bob 190, there is provided a concave groove 198b extending in a circle around the axially facing side of the second bob 191. Shear waves emanating from the concave groove 198a of the first bob 190 are focused to a region between the two bobs 190, 191. Shear waves emanating from the concave groove 198b of the second bob 191 are focused to a region between the two bobs 190, 191.

FIG. 35, in accordance with the techniques of this disclosure, illustrates an arrangement in accordance with the techniques of this disclosure in which two opposing discs 200, 201 are mounted on respective shafts 207, 208 and configured to vibrate torsionally. A first face 204 of the first disc 200 faces a second face 206 of the second disc 201. Shear waves that propagate across the gap distance between the first face 204 and the second face 206 may interfere with each other constructively or destructively.

FIG. 36, in accordance with the techniques of this disclosure, illustrates an arrangement in which a first bob 210 is located on a first torsionally vibrating shaft 217 and a second bob 211 is located on a second torsionally vibrating shaft 218. The shafts 217, 218 are axially aligned and vibrate about the same axis. In between the two bobs 210, 211 there is a gap. The outmost radial extent of the first bob 210 is greater than the outmost radial extent of the second bob 211. During torsional vibration of the shaft 218, shear waves emanate out radially from the outer cylindrical surface 216 of the second bob 201. In addition, shear waves emanate out axially from an axial-facing surface 214 of the first bob 201 across the gap between the first and second bobs 210, 211. Such shear waves may interfere with each other constructively or destructively. Because of the gap between the first bob 210 and the second bob 211, the contributory of any constructive interference is reduced, favourably permitting net destructive interference.

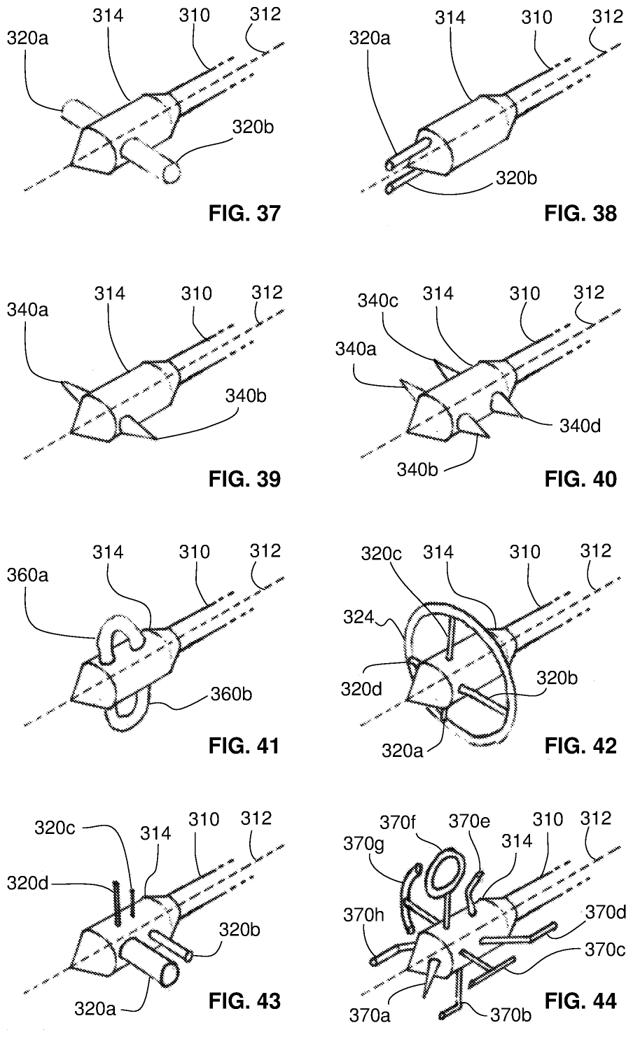

FIG. 37 illustrates a vibratory transducer in which two elongate members 320a, 320b are connected to the second axial section of the bob 314. The two elongate members 320a, 320b are cylindrical and have the same radius that is constant along their lengths. A first of the elongate members 320a is diametrically spaced around the bob 314 from the other elongate member 320b. Each elongate member 320a, 320b extends outwards from the bob 314 in a radial direction that is perpendicular to the axis 312 of the shaft 310. Either or both of the first and second elongate members 320a, 320b may provide a surface with which constructive or destructive interference may arise with waves emanating from the conical surface of the bob 314.

FIG. 38 illustrates a vibratory transducer in which two elongate members 320a, 320b are connected to the third axial section of the bob 314. Each elongate member 320a, 320b is aligned along a respective axis that is parallel to the axis 312 of the shaft 310 but offset radially from the axis 312 of the shaft 310. The two elongate members 320a, 320b are cylindrical and have the same radius that is constant along their lengths. A first of the elongate members 320a is diametrically spaced around the third axial section of the bob 314 from the other elongate member 320b. Either or both of the first and second elongate members 320a, 320b may provide a surface with which constructive or destructive interference may arise with waves emanating from the conical surface of the bob 314 or with waves emanating from each other.

FIG. 39 illustrates a vibratory transducer in which two elongate members 340a, 340b are connected to the second axial section of the bob 314. The two elongate members 340a, 340b are conical. A first of the elongate members 340a is diametrically spaced around the bob 314 from the other elongate member 340b. Each elongate member 340a, 340b extends outwards from the bob 314 in a radial direction that is perpendicular to the axis 312 of the shaft 310. Each elongate member 340a, 340b has a maximum radius at a proximal end, at which it is connected to the second axial section of the bob 314. Either or both of the first and second elongate members 340a, 340b may provide a surface with which constructive or destructive interference may arise with waves emanating from the cylindrical surface of the bob 314.

FIG. 40 illustrates a vibratory transducer in which four elongate members 340a, 340b, 340c, 340d are connected to the second axial section of the bob 314. The four elongate members 340a, 340b, 340c, 340d are conical. A first of the elongate members 340a is diametrically spaced around the bob 314 from a second elongate member 340b but at the same axial location along the axis 312 as the second elongate member 340b. A third of the elongate members 340c is diametrically spaced around the bob 314 from a fourth elongate member 340d but at the same axial location along the axis 312 as the fourth elongate member 340d. The first and second elongate members 320a, 320b are axially spaced along the axis 312 from the third and fourth elongate members 340c, 340d respectively. Each elongate member 340a, 340b, 340c, 340d has a maximum radius at a proximal end, at which it is connected to the second axial section of the bob 314. Any or all of the first to fourth elongate members 340a, 340b, 340c, 340d may provide a surface with which constructive or destructive interference may arise with waves emanating from the cylindrical surface of the bob 314 or with waves emanating from each other.

FIG. 41 illustrates a vibratory transducer in which two elongate members 360a, 360b are connected to the second axial section of the bob 314. The two elongate members 360a, 360b each have the form of a semitorus. A first elongate member 360a is curved and connects to the bob 314 at two axially spaced locations and has a circular cross section along the curved axis through elongate member 360a. A second elongate member 360b is curved and connects to the bob 314 at two axially spaced locations that are diametrically spaced around the bob from the locations where the first elongate member 360a connects to the bob 314. The second elongate member also has a circular cross section along the curved axis through elongate member 360b. Either or both of the first and second elongate members 360a, 360b may provide a surface with which constructive or destructive interference may arise with waves emanating from the cylindrical surface of the bob 314, whether at the approximately radially aligned base portions that are proximal to the surface of the bob 314 or at the approximately axially aligned ‘loop’ portions that are distal to the surface of the bob 314.

FIG. 42 illustrates a vibratory transducer in which four elongate members 320a, 320b, 320c, 320d are connected to the second axial section of the bob 314. Each of the four elongate members 320a, 320b, 320c, 320d are cylindrical with the same constant radius and extend in a radial direction from the bob 314 from locations distributed around a circumference of the second axial section at 90° from neighbouring elongate members. Each distal end of the four elongate members 320a, 320b, 320c, 320d is connected to a fifth elongate member 324 that encircles the bob 314 and has the form of a torus or ring. Any or all of the first to fourth elongate members 320a, 320b, 320c, 320d, or the fifth elongate member 324 that has the form of a torus or ring, may provide a surface with which constructive or destructive interference may arise with waves emanating from the cylindrical surface of the bob 314 or with waves emanating from each other.

FIG. 43 illustrates a vibratory transducer in which four elongate members 320a, 320b, 320c, 320d are connected to the second axial section of the bob 314. The four elongate members 320a, 320b, 320c, 320d are cylindrical but have different radiuses. A first elongate member 320a has a largest radius. A second elongate member 320b has a radius that is smaller than the radius of the first elongate member 320a. A third elongate member 320c has a radius that is smaller than the radius of the second elongate member 320b. A fourth elongate member 320d has a radius that is smaller than the radius of the third elongate member 320c. The first elongate member 320a and second elongate member 320b each extend radially outward from the second axial section of the bob 314 from respective locations that are axially offset from each other but otherwise from the same circumferential location. The third elongate member 320c and fourth elongate member 320d each extend radially outward from the second axial section of the bob 314 from respective locations that are axially offset from each other but otherwise from the same circumferential location. The first and second elongate members 320a, 320b are spaced around the circumference of the bob 314 from the third and fourth elongate members 320c, 320d by 90°. Any or all of the first to fourth elongate members 320a, 320b, 320c, 320d may provide a surface with which constructive or destructive interference may arise with waves emanating from the cylindrical surface of the bob 314 or with waves emanating from each other.

FIG. 44 illustrates a vibratory transducer in which eight elongate members 370a, 370b, 370c, 370d, 370e, 370f, 370g, 370h are connected to the bob 314.

A first elongate member 370a is connected to the third axial section of the bob 314 and has the form of a cone extending in a direction that oblique to the axis 312 of the shaft 310. The first elongate member 370a may provide a surface with constructive or destructive interference may arise with waves emanating from the conical surface of the bob 314.

A second elongate member 370b is connected to the second axial section of the bob 314 and extends, from its proximal end, radially outward from the bob 314. The second elongate member 370b is not straight-it comprises two straight sections that connect at a right angle to form an ‘L’-shaped member. The distal section of the second elongate member 370b extends in a direction parallel to the axis 312. Either or both of the proximal and distal sections of the second elongate 370b member may provide a surface with which constructive or destructive interference may arise with waves emanating from the cylindrical surface of the bob 314 or with waves emanating from each other.

The third elongate member 370c is connected to the second axial section of the bob 314 and has the form of a ‘T’-shape. A proximal section of the third elongate member 370c is connected to the bob 314 and extends radially outward from the bob 314. A distal section of the third elongate member 370c is connected to a distal end of the proximal section of the third elongate member 370c and extends in a direction parallel to the axis 312, i.e. perpendicular to the direction of the proximal section. Either or both of the proximal and distal sections of the third elongate member 370c may provide a surface with which constructive or destructive interference may arise with waves emanating from the cylindrical surface of the bob 314 or with waves emanating from each other or any other elongate member.

The fourth elongate member 370d is connected to the second axial section of the bob 314 and comprises a proximal section and a distal section, the proximal section connected to the bob 314 and extending in an oblique direction relative to the axis 312 of the shaft 310, an end of the distal section connected to a distal end of the proximal section, the distal section extending in a direction parallel to the axis 312. Either or both of the proximal and distal sections of the fourth elongate member 370d may provide a surface with which constructive or destructive interference may arise with waves emanating from the cylindrical surface of the bob 314 or with waves emanating from each other or any other elongate member.

The fifth elongate member 370e is connected to the second axial section of the bob 314. The fifth elongate member 370e is not straight but extends outward from the bob 314 in a broadly radial direction but it has a broadly circular cross section along its length. The fifth elongate member 370e may provide a surface with which constructive or destructive interference may arise with waves emanating from the cylindrical surface of the bob 314 or with waves emanating from any other elongate member.

The sixth elongate member 370f is connected to the second axial section of the bob 314 and extends radially outward from the bob 314. At a distal end of the sixth elongate member 370f, a further elongate member is connected, the further elongate member having the form of a closed loop and specifically a torus. Either or both of the proximal and distal sections of the fifth elongate member 370e may provide a surface with which constructive or destructive interference may arise with waves emanating from the cylindrical surface of the bob 314 or with waves emanating from each other or any other elongate member.

The seventh elongate member 370g is connected to the second axial section of the bob 314 and extends radially outward from the bob 314. At a distal end of the seventh elongate member 370g, a further elongate member is connected, the further elongate member being curved in a circumferential about an arc that is concentric with the circumference of the second axial section of the bob 314. Either or both of the proximal and distal sections of the seventh elongate member 370g may provide a surface with which constructive or destructive interference may arise with waves emanating from the cylindrical surface of the bob 314 or with waves emanating from each other or any other elongate member.

The eighth elongate member 370h is connected to the third axial section of the bob 314 and comprises a proximal section and a distal section, the proximal section connected to the bob 314 and extending outward from the surface of the third axial section in an oblique direction relative to the axis 312 of the shaft 310, an end of the distal section connected to a distal end of the proximal section, the distal section extending in a direction parallel to the axis 312.

Either or both of the proximal and distal sections of the eight elongate member 370g may provide a surface with which constructive or destructive interference may arise with waves emanating from the cylindrical surface or conical surface of the bob 314 or with waves emanating from each other or any other elongate member.

It is noted that some embodiments may include only a subset of the elongate members illustrated in FIGS. 37 to 44, such as one or more of the many diverse elongate members illustrated in FIG. 44. The skilled reader will recognise in particular that FIG. 44 illustrates a wide range of possible elongate members in a single device and that practical implementations might not include all of the elongate members illustrated in FIG. 44.

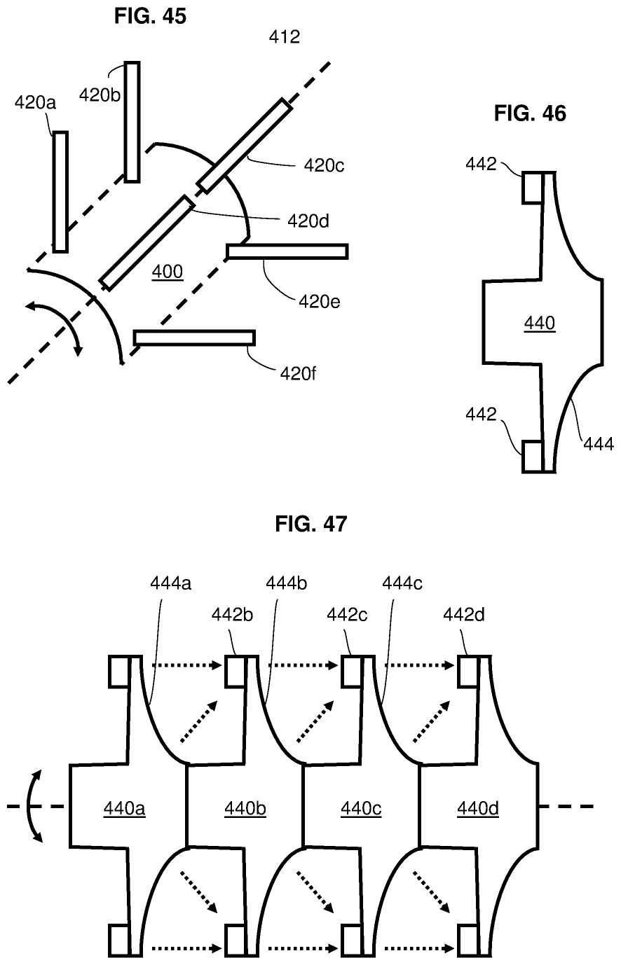

FIG. 45 illustrates a configuration of a portion of a vibratory transducer in accordance with the techniques of this disclosure, in which elongate members 420a, 420b, 420c, 420d, 420e, 420f are connected to a shaft or bob 400 configured to vibrate torsionally about an axis 412. The elongate members 420a, 420b, 420c, 420d, 420e, 420f are cylindrical. The first elongate member 420a and second elongate member 420b each extend radially outward from the shaft or bob 400 from respective locations that are axially offset from each other but otherwise from the same circumferential location. The third elongate member 420c and fourth elongate member 420d each extend radially outward from the shaft or bob 400 from respective locations that are axially offset from each other but otherwise from the same circumferential location. The fifth elongate member 420e and sixth elongate member 420f each extend radially outward from the shaft or bob 400 from respective locations that are axially offset from each other but otherwise from the same circumferential location. The first and second elongate members 420a, 420b are spaced around the circumference of shaft or bob 400 from the third and fourth elongate members 420c, 420d by 45°. The fifth and sixth elongate members 420a, 420b are spaced around the circumference of shaft or bob 400 from the third and fourth elongate members 420c, 420d by 45° and from the first and second elongate members 420a, 420b by 90°. Any or all of the first to sixth elongate members 420a, 420b, 420c, 420d, 420e, 420f may provide a surface with which constructive or destructive interference may arise with waves emanating from the cylindrical surface of the shaft or bob 400, or with waves emanating from others of the elongate members.

In addition, adjacent pairs of elongate members, such as the first and second elongate members 420a, 420b, are spaced axially from each other such that shear waves emanating from them destructively interfere. Shear waves emanating from the first elongate member 420a are, in the neighbourhood of the second elongate member 420b, out of phase with shear waves emanating from the second elongate member 420b. Shear waves emanating from the second elongate member 420b are, in the neighbourhood of the first elongate member 420a, out of phase with shear waves emanating from the first elongate member 420a. The first and second elongate members may act as mutual radiators and detectors. Similar destructive interference may occur between shear waves emanating between the third and fourth elongate members 420c, 420d and between shear waves emanating between the fifth and sixth elongate members 420e, 420f. In accordance with the techniques of this disclosure, similar destructive interference (or constructive interference if desired) may occur between any coupled pair of elongate remembers depending on geometry and shear wave propagation characteristics (propagation depth, wavelength etc.).

In another configuration in accordance with the techniques of this disclosure (with which optionally net constructive or destructive interference may occur when under vibration), a vibratory transducer includes a shaft and a plurality of elongate members. If the vibratory transducer comprises a bob, then the elongate members may be connected to the vibratory transducer at the bob. Alternatively or additionally, the vibratory transducer may comprise elongate members connected to the vibratory transducer at the shaft. A plurality of elongate members are spaced around the circumference of the shaft or bob, extending outward from the shaft or bob in a wholly radial direction or in a direction with a radial component and an axial component, or in a wholly axial direction (that is not colinear with a longitudinal axis of the shaft/bob). The plurality of elongate members may be distributed evenly around the circumference, which may mitigate or avoid any disturbance in the centre of mass relative to the longitudinal axis, or may be distributed unevenly around the circumference. The plurality of elongate members may be connected to the shaft or bob at the same axial position along the length of the shaft or bob, or may be connected at different axial positions, e.g. in a helical pattern around the exterior surface of the shaft or bob.

If the plurality of elongate members extend from the shaft or bob in a wholly or partially axial direction, then the elongate members may include spacing supports from an outer surface of the shaft or bob to provide a radial offset to the elongate members. Alternatively, the plurality of elongate members may extend from an end of the shaft or bob, such as distributed in a circle around the longitudinal axis and extending from the end of the shaft or bob. The end of the shaft or bob may be flat, curved, conical or have some other profile.

The elongate members may have a width and half width such that geometric damping (monopole behaviour) may take place around the elongate members during vibration of the vibratory transducer. The vibration of the vibratory transducer may be torsional about the longitudinal axis of the shaft.

The vibratory transducer may comprise two or more elongate members, such as three, four, five, six, seven, eight, nine, ten or more elongate members. The elongate members may have constant cross sections along their lengths (such as a cylindrical elongate member or an elongate member having a square/rectangular, rounded square/rectangular (e.g. in the form of a superellipse), triangular or elliptical cross section) or may have varying cross sections shapes or sizes along their lengths, such as i) a cone of linearly decreasing cross-section area with increasing distance from the shaft or bob, or ii) stepped variations in size or shape (e.g. stepped decrements in size) with increasing distance from the shaft or bob.

In a particular configuration, a vibratory transducer comprises a shaft configured for torsional vibration, the shaft having a proximal end at which the vibrations are driven and a distal end. At or close to the distal end of the shaft (e.g. nearer to the distal end than the proximal end, or within a final quarter of the fluid-contacting length of the shaft, or within a final tenth of the fluid-contacting length of the shaft, or within a final twentieth of the fluid-contacting length of the shaft), a plurality of elongate members extend radially outward from the shaft at a common axial position along the length of the shaft. Eight elongate members are distributed evenly around the circumference of the shaft at 45° increments. In another particular configuration, a bob is present at the distal end of the shaft and the eight elongate members extend radially outward from bob. Other configurations comprise more or fewer elongate members, distributed evenly around the shaft/bob or distributed unevenly. For example, one configuration comprises six elongate members distributed evenly around the circumference of the shaft.

In another particular configuration, a vibratory transducer includes a shaft and a plurality of elongate members that are aligned axially with the longitudinal axis of the shaft but are not colinear with the longitudinal axis of the shaft. At a distal end of the shaft there is a bob. The bob has the form of a cylinder that is coaxial with the longitudinal axis of the shaft but has a larger radius than the shaft. The plurality of elongate members extend axially outward from an end of the bob, each connected to the end of the bob at the same radial offset from the longitudinal axis and distributed evenly around the longitudinal axis. The plurality of elongate members comprise eight elongate members that are distributed evenly around the longitudinal axis at 45° increments. The vibratory transducer is configured to vibrate torsionally about the longitudinal axis. Other configurations comprise more or fewer elongate members, distributed evenly around the longitudinal axis or distributed unevenly. For example, one configuration comprises six elongate members distributed evenly around the longitudinal axis.

FIGS. 46 and 47 illustrate a configuration in accordance with the techniques of this disclosure employing an advantageous modularity.

FIG. 46 illustrates a cross section of segment 440 of a vibratory transducer through an axis of torsional vibration, in which an axially symmetric flange extends outward from a shaft, the flange provided with a convex surface 444 on one axial side of the flange and a detector surface 442 (shown as rectangular in cross section purely by way of example) is provided on a second side of the flange. This segment 440 may be axially aligned with other identical or similar segments such that the convex surface 444 may generate focused shear waves at a detector of an adjacent segment and the detector 442 may receive focused shear waves from a convex surface of an adjacent segment.

FIG. 47 illustrates a cross section of a vibratory transducer in which four segments 440a, 440b, 440c, 440d identical to the segment shown in FIG. 46 are aligned axially and adjacently and configured to vibrate torsionally about a common axis. The convex surface 444a of the first segment 440a provides focused shear waves to the detector surface 442b of the second segment 440b. The convex surface 444b of the second segment 440b provides focused shear waves to the detector surface 442c of the third segment 440c. The convex surface 444c of the third segment 440c provides focused shear waves to the detector surface 442d of the fourth segment 440d. A modular configuration of repeated segments such as shown in FIG. 47 may provide advantages in manufacturing and in the design of a vibratory transducer for specific operational requirements. In particular, a sensitivity of a transducer may be varied by increasing or reducing a number of segments, without having to vary a segment's geometry.

Both radiator and detector surfaces may be surfaces of elongate members that may employ geometric damping.

Some explanation of geometric damping and elongate members employing geometric damping is presented below, wherein a purely viscous fluid is first considered, followed by a viscoelastic fluid.

From FIG. 1 and Equation 7, for a purely viscous fluid, the propagation depth of a shear wave is given by the expression:

$${x}_{0 , μ} = \sqrt{\frac{2 {μ}^{′}}{ω ρ}} , \qquad Equation 18$$

The decreasing amplitude of the wave caused by the viscosity creates a shear stress, τ_{Sμ}, at the vibrating surface that is a product of the rate of change of velocity at the surface (i.e. the shear rate \dot{γ}) and the fluid viscosity, μ′, given by the following expression:

$${τ}_{S μ} = \dot{γ} {μ}^{′} . \qquad Equation 19$$

The shear rate \dot{γ}_{μ}, at the oscillating surface caused by wave attenuation can be determined by differentiating an expression for the wave velocity with respect to the distance from the surface, and evaluating the expression at the surface, leading to the following expression:

$${\dot{γ}}_{μ} = - {V}_{0} α = - \frac{{V}_{0}}{{δ}_{μ}} = - {V}_{0} \sqrt{\frac{ω ρ}{2 {μ}^{′}}} , \qquad Equation 20$$

where V₀ is a shear velocity at the oscillating surface.

The shear rate due to viscous attenuation is proportional to the square root of the frequency and proportional to the square root of the density and proportional to the square root of the reciprocal of the viscosity.

Therefore, the shear stress at the surface (which is a product of the viscosity and shear rate at the surface) is non-linear:

$${τ}_{S μ} = - {V}_{0} \sqrt{\frac{ωρ}{2 {μ}^{′}}} {μ}^{′} = - {V}_{0} \sqrt{\frac{{μ}^{′} ω ρ}{2}} . \qquad Equation 21$$

In addition to viscous effects, a fluid may show elastic behaviour, which is dependent on the storage modulus, G′. The presence of G′ reduces the loss tangent, tan Δ.

As discussed above, for a purely viscous fluid, tan Δ=∞. With increased elastic behaviour the fluid becomes less lossy, allowing the wave to propagate further into the fluid. Taking elasticity into account, the propagation depth is given by Equation 7, in which 1/(sin(Δ/2)√(2 sin Δ)) is a quantity that scales the propagation depth due to the fluid's elastic behaviour. This quantity is also equal to 1/√(sin Δ(1−cos Δ)).

For a purely viscous fluid, A is equal to 90° and so the scaling quantity given by

$$1 / ( sin ( \frac{Δ}{2} ) √ ( 2 sin Δ ) )$$

(or equivalently 1/√(sin Δ(1−cos √))) is equal to 1 and the purely viscous propagation depth, δ_{μ}, is recovered. Thus it is appropriate to refer to a viscoelastic propagation depth using this expression even for fluids that exhibit little or no viscoelasticity. For values of Δ less than 90°, the scaling quantity is greater than 1 and so the propagation depth is increased relative to the purely viscous propagation depth.

The shear rate at the oscillating surface due to both viscosity and elasticity is given by the expression:

$${\dot{γ}}_{μ {G}^{′}} = - \frac{{V}_{0}}{{δ}_{μ , {G}^{′}}} = - {V}_{0} sin \frac{Δ}{2} \sqrt{\frac{ρω sin Δ}{{μ}^{′}}} . \qquad Equation 22$$

The shear stress at the oscillating surface due to both viscosity and elasticity is given by the expression:

$${τ}_{S μ G ′} = {\dot{γ}}_{μ {G}^{′}} {μ}^{′} = - {V}_{0} sin \frac{Δ}{2} \sqrt{ρ ω {μ}^{′} sin Δ} . \qquad Equation 23$$

The shear stress is a non-linear function of the fluid viscosity, fluid density, frequency and storage modulus (via the loss tangent). As the elasticity G′ increases, tan A decreases, Δ decreases from a maximum of π/2, and both sin A and sin Δ/2 decrease, and so the damping shear stress is reduced as the elasticity G′ increases. This explains why viscoelastic fluids show reduced damping compared with Newtonian fluids of ‘similar’ viscosity.

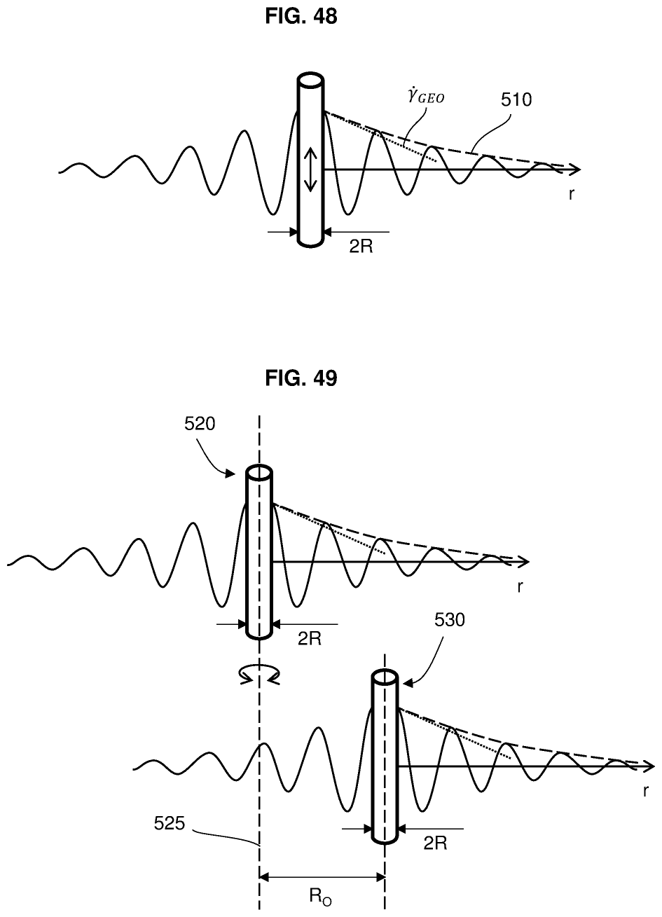

FIG. 48 illustrates a shear wave radially propagating in a viscoelastic fluid from a curved surface with radius R. While a viscoelastic fluid is relatively lossless over a short distance, FIG. 48 shows the amplitude decreasing because the potential energy of each wave peak has to be maintained as the radial distance increases, the energy spread out over an increasing circumferential length (2πr). A line 510 of constant potential energy is shown in FIG. 48. The distribution of energy over the increasing circumferential length leads to a reduced energy per unit volume and therefore a smaller peak height. This attenuation of amplitude due to geometric considerations appears similar to damping although it does not itself dissipate energy.

The change in height causes a decrease in velocity that is proportional to 1/r and this change of velocity gives rise to a shear rate, \dot{γ}_{RAD}, which combines with the viscosity to create a shear stress, τ_{RAD}, which will have a component in phase with the surface velocity, which causes energy to be dissipated. This effect is termed ‘geometric damping’ herein.

If, instead of a planar surface oscillating as in FIG. 1, a cylindrical surface of radius R is oscillating, an expression for the velocity of a radial shear wave at a location r from the central axis of the cylinder may be obtained and itself differentiated with respect to r to obtain the radial shear rate:

$${\dot{γ}}_{r} = - {V}_{r} ( \frac{1}{r} + \sqrt{\frac{ω ρ}{μ}} {e}^{i ϕ} ) = - {V}_{r} ( \frac{1}{r} + \frac{1}{{δ}_{μ G ′}} ) \qquad Equation 24$$

where Vᵣ is a shear velocity at the surface, ϕ is a phase adjustment angle given by tan⁻¹ (β/α) and equal to π/2-λ/2, where β is the wavenumber of the propagating wave (i.e. 2π/λ, where λ is a wavelength of the propagating wave).

The shear stress at the cylindrical surface, where r=R, is then given by the expression:

$$\begin{matrix}{τ}_{S μ {G}^{′}} = {\dot{γ}}_{μ {G}^{′}} {μ}^{′} = - {V}_{r} ( \frac{1}{R} + \frac{1}{{δ}_{μ {G}^{′}}} ) {μ}^{′} \\ = - {V}_{r} ( \frac{1}{R} + \sqrt{\frac{ω ρ}{\vert μ \vert}} \cdot {e}^{i ϕ} )\end{matrix} \qquad Equation 25$$

The 1/R term is an in-phase shear gradient. The shear rate for this component is in phase with velocity for any degree of viscoelasticity. The 1/δ_{μG}, term is an out-of-phase shear gradient. The shear rate for this component is out of phase by, which depends on the degree of viscoelasticity. The phase adjustment angle represents the angle between shear stress and velocity. A shear stress that is in phase with velocity dissipates energy.

For values of R much less than δ_{μG}′, the in-phase portion dominates and the shear rate becomes less dependent or even independent of viscosity, density, frequency and storage modulus (through tan Δ/2, which is a function of G″). If 1/δ_{μG′} is negligible compared with 1/R, then the shear stress at the cylindrical surface is given by the expression:

$${τ}_{S μ G ′} = - \frac{{V}_{r} {μ}^{′}}{R} . \qquad Equation 26$$

For values of R much greater than δ_{μG′}, the out-of-phase portion dominates and the shear rate becomes increasingly dependent on non-linear functions of viscosity, density, frequency and storage modulus. If 1/R is negligible compared with 1/δ_{μG}′, then the shear stress at the cylindrical surface is given by the expression:

$${τ}_{S μ G ′} = - \frac{{V}_{r} {μ}^{′}}{{δ}_{μ {G}^{′}}} = - {V}_{r} sin \frac{Δ}{2} \sqrt{ρ ω {μ}^{′} sin Δ} . \qquad Equation 27$$

A critical value of R is at Rₒₙₛₑₜ=δ_{μG′} because this represents a cross-over point for the value of R where the shear stress due to the 1/R term becomes larger than the shear stress due to the 1/δ_{μG}, term. In accordance with the techniques described herein, this may be considered the onset of geometric damping.

The dependence of the shear stress on non-linear functions of viscosity, density, frequency and storage modulus is reduced further when R<δ_{μG′}. It may be considered that, if the cylinder radius is less than half of the viscoelastic propagation depth, then geometric damping begins to dominate, i.e. where R<Rₒₙₛₑₜ/2. In other words, R_{geo}=Rₒₙₛₑₜ/2, where R_{geo} may be understood to be a cylinder radius that defines a regime under which geometric damping can be assumed to dominate and define the damping behaviour.

For the measurement of physical properties of a fluid, parameters can be selected to provide improved linearity of fluid loading factors, such as a fluid damping factor, C_{F}, a stiffness loading factor, K_{F}, and an inertial loading factor, J_{F}. If R=δ_{μG′}, then an expression for the fluid viscosity at which the onset of geometric damping occurs is given by:

$${μ}_{onset}^{′} = {R}^{2} ωρ \cdot {sin}^{2} \frac{Δ}{2} \cdot sin Δ = {R}^{2} 2 π f ρ \cdot {sin}^{2} \frac{Δ}{2} \cdot sin Δ , \qquad Equation 28$$

$$where$$

$$ω = 2 π f .$$

For example, given a cylindrical element having a radius R of 2 mm vibrating in a purely viscous fluid (sin (Δ/2)√(2 sin Δ)=1) at a frequency of 5 kHz, the fluid having a density ρ of 1000 kg/m³, then an appropriate choice of fluid viscosity, in units of Pa·s, at which R=Rₒₙₛₑₜ is given by:

$${μ}_{onset}^{′} = {2}^{2} \cdot π \cdot 5000 \cdot 1000 = 62$$

For geometric damping to begin to dominate (i.e. where R_{geo}=Rₒₙₛₑₜ/2), the required viscosity is four times higher, i.e.:

$${μ}_{geo}^{′} = {( 2 \cdot 2 )}^{2} \cdot π \cdot 5000 \cdot 1000 = 250 .$$

Similarly, a radius of a vibrating element and/or a frequency of vibration can be selected to exploit geometric damping for a given working range of viscosity and density according to operational requirements.

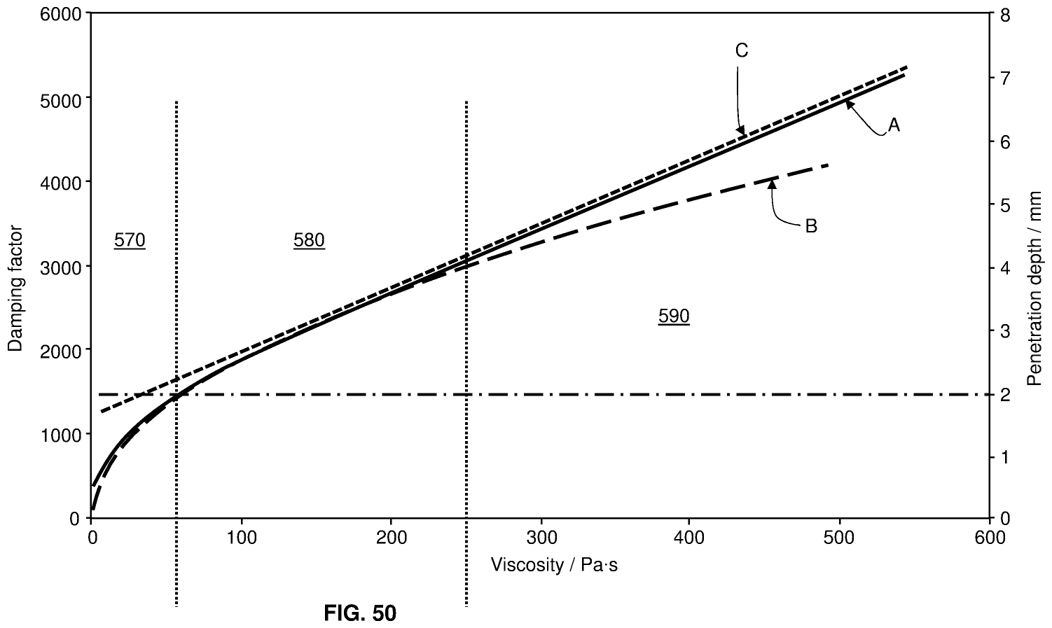

FIG. 50 shows a graph of measured damping factor for a heavy mineral oil using a vibrating cylinder of radius 2 mm at a frequency of 5 kHz, the fluid having a density ρ of 1000 kg/m³, the damping factor measured over a range of viscosity values (obtained by applying heat to the heavy mineral oil). The measured damping factor is indicated by the solid line denoted by ‘A’ in FIG. 4. The graph also shows a plot of the calculated damping factor if the damping is described by non-geometric damping, i.e. if 1/R is negligible compared with 1/δ_{μG}′. This line is denoted by ‘B’ in FIG. 4. The graph also shows a plot of the calculated damping factor if the damping is described by geometric damping, i.e. if 1/δ_{μG}′, is negligible compared with 1/R. This line is denoted by ‘C’ in FIG. 4.

FIG. 50 demonstrates that, while the viscosity is relatively low, i.e. below approximately 60 Pa·s, the radius of the vibrating element is larger than the viscous penetration depth and the shear rate is a non-linear function of viscosity. Where the viscosity is above approximately 60 Pa·s, shear wave attenuation begins to be described by geometric damping and the damping factor is seen to become increasingly linear.

FIG. 4 identifies three regions. A first region denoted by reference 570 is a non-linear region and covers viscosity values below μₒₙₛₑₜ (62 Pa·s as determined above). A second region denoted by reference 580 is a linear-transition region and covers viscosity values between μₒₙₛₑₜ and μ_{geo} (250 Pa·s as determined above). A third region denoted by reference 590 is a fully linear region and covers viscosity values above μ_{geo}. Operating in the second region 580 provides improved linearity compared with operating in the first region 570. Operating in the third region 590 provides improved linearity compared with operating in the second region 580.

Expressions for the fluid damping factor, C_{F}, stiffness loading factor, K_{F}, and inertial loading factor, J_{F}, for a vibrating cylinder of sufficiently wide radius that geometric damping is negligible are given by the following expressions:

$${C}_{F} = ( A \cdot \frac{{R}_{G}^{2}}{{δ}_{μ , G ′}} ) {μ}^{′} = ( A \cdot {R}_{G}^{2} \frac{1}{sin \frac{Δ}{2}} \sqrt{\frac{2 {μ}^{′}}{ρ ω sin Δ}} ) {μ}^{′} \qquad Equation 29$$

$${K}_{F} = ( A \cdot \frac{{R}_{G}^{2}}{{δ}_{μ , G ′}} ) {G}^{′} = ( A \cdot {R}_{G}^{2} \frac{1}{sin \frac{Δ}{2}} \sqrt{\frac{2 {μ}^{′}}{ρ ω sin Δ}} ) {G}^{′} \qquad Equation 30$$

$${J}_{F} = ( A \cdot {R}_{G}^{2} \cdot {δ}_{μ , G ′} ) ρ = ( A \cdot {R}_{G}^{2} \frac{1}{sin \frac{Δ}{2}} \sqrt{\frac{2 {μ}^{′}}{ρ ω sin Δ}} ) ρ \qquad Equation 31$$

In these expressions, each of the factors has a non-linear dependency on μ′, G′, or ρ due to the presence of these terms (or quantities that are a function of these terms, such as Δ and its dependency on G′) inside the parentheses.

If a vibrating cylinder has a sufficiently small radius that non-geometric damping is negligible, then expressions for C_{F}, K_{F}, and J_{F} are given by:

$${C}_{F} = ( A \cdot \frac{{R}_{G}^{2}}{R} ) {μ}^{′} \qquad Equation 32$$

$${K}_{F} = ( A \cdot \frac{{R}_{G}^{2}}{R} ) {G}^{′} \qquad Equation 33$$

$${J}_{F} = ( A \cdot {R}_{G}^{2} \cdot R ) ρ \qquad Equation 34$$

It can be seen from these expressions where non-geometric damping is negligible that the fluid damping factor, C_{F}, stiffness loading factor, K_{F}, and inertial loading factor, J_{F}, no longer have any non-linear dependency on μ′, G′, or ρ. The quantities C_{F}, K_{F}, and J_{F} each respectively directly proportional to μ′, G′, and ρ, with a constant of proportionality that depends only on geometric parameters.

Improved linearity of these fluid loading factors may be advantageous. A mechanical system may have damping C, stiffness K, and inertia J. These determine the vibration frequency ω and the Q-factor of the system through the following equations:

$$ω = \sqrt{K / J} \qquad Equation 35$$

$$Q = \frac{\sqrt{K \cdot J}}{C} \qquad Equation 36$$

When the system is vibrating in air or a vacuum, these mechanical factors can be declared C₀, K₀, and J₀. When the system is vibrating a fluid, the physical properties of the fluid ‘load’ these factors by amounts C_{F}, K_{F} and J_{F} respectively.

The overall values for damping, stiffness, and inertia factors of the system taking into account the fluid loading may be expressed as:

$$C = {C}_{0} + {C}_{F} \qquad Equation 37$$

$$K = {K}_{0} + {K}_{F} \qquad Equation 38$$

$$J = {J}_{0} + {J}_{F} \qquad Equation 39$$

The overall values for C, K, and J are related to the above equations for frequency and Q-factor, which are readily measurable. The physical properties of the fluid may be determined based on the contributions of C_{F}, K_{F} and J_{F} to the vibrational behaviour. As discussed below, the techniques of this disclosure may provide simple linear relationships between C_{F}, K_{F} and J_{F} and physical properties of interest, such as density p, viscosity μ′, and storage modulus G′.

In these expressions, A is the fluid-contacting surface area of the cylindrical element and R_{G} is a ‘radius of gyration’ of the element, equal to the radius R of the cylindrical element when the cylindrical element vibrates torsionally about its axis.

In considering the effect of a moment of inertia on rotational motion of a body, the radius of gyration is as the radial distance to a point which would have a moment of inertia the same as the body's actual distribution of mass, if the total mass of the body were concentrated there. The term ‘radius of gyration’ in the present disclosure is a generalisation of this concept to consider torsional factors torsional other than moments of inertia.

In the case of the damping factor, C_{F}, the radius of gyration represents the radial distance to a point which would have the same damping effect as the body's actual damping effect if the damping were concentrated at that point.

In the case of the stiffness loading factor, K_{F}, the radius of gyration represents the radial distance to a point which would have the same stiffness loading effect as the body's actual stiffness loading effect if the stiffness loading were concentrated at that point.

In the case of the inertial loading factor, K_{F}, the radius of gyration represents the radial distance to a point which would have the same inertial loading effect as the body's actual inertial loading effect if the inertial loading were concentrated at that point.

Thus the radius of gyration defined more generally in this disclosure represents a convenient measure of the radial effect of these loading factors. The radius of gyration will be defined by the specific geometry of the cylindrical element, but will generally be assumed to have upper and lower bounds defined by a maximum and minimum radial extent of the cylindrical element from the axis of rotation, and be equal to the radius R of the cylindrical element when the cylindrical element vibrates torsionally about its axis, since all of the surface loading takes place at the cylindrical surface, all at distance R from the axis.

FIG. 49 shows a first cylinder 520 of radius R rotating torsionally about an axis 25 lengthwise through centre of the cylinder. The cylinder has a length I rotating about its axis, where the length is sufficiently long that the area (πR²) of an end portion is small compared to an area of the curved sides (2TRl) and where R<<δ_{μG}, and R=R_{G}, the damping factor, C_{F}, stiffness loading factor, K_{F}, and inertial loading factor, J_{F}, may be written as:

$${C}_{F} = A R {μ}^{′} = ( 2 π {R}^{2} l ) {μ}^{′} \qquad Equation 40$$

$${K}_{F} = A R {G}^{′} = ( 2 π {R}^{2} l ) {G}^{′} \qquad Equation 41$$

$${J}_{F} = {AR}^{3} ρ = ( 2 π {R}^{4} l ) ρ . \qquad Equation 42$$

While geometric damping through the propagation of radial waves brings advantages through a dependency on geometric parameters rather than fluid properties, the conditions for geometric damping encourage the use of a cylindrical element with a small radius, which results in a small active surface area. The small R² term in the damping, stiffness and inertial loading factors means that the damping, or elastic or inertial loading factors from torsional vibration are small, even at high viscosities.

FIG. 49 also shows a second cylinder 530 having the same size as the first cylinder 520, wherein the second cylinder 530 is displaced perpendicularly from the axis of the axis 525 of the first cylinder 520 by an offset radius Ro that is greater than R. The second cylinder 530 is also vibrated torsionally about the axis 525 of the first cylinder 520. This has the effect of changing the radius of gyration R_{G} from R (the distance from the cylindrical surface to the axis 525) to Ro (the radial offset of the cylinder as a whole).

If the equations provided above for the damping factor, C_{F}, stiffness loading factor, K_{F}, and inertial loading factor, J_{F}, under geometric damping hold (i.e. negligible non-geometric damping), then the equations may be expressed as:

$${C}_{F} = ( A \cdot \frac{{R}_{O}^{2}}{R} ) {μ}^{′} = ( \frac{2 π R l {R}_{O}^{2}}{R} ) {μ}^{′} = ( 2 π l {R}_{O}^{2} ) {μ}^{′} \qquad Equation 43$$

$${K}_{F} = ( A \cdot \frac{{R}_{O}^{2}}{R} ) {G}^{′} = ( \frac{2 π R l {R}_{O}^{2}}{R} ) {G}^{′} = ( 2 π l {R}_{O}^{2} ) {G}^{′} \qquad Equation 44$$

$${J}_{F} = ( A \cdot {R}_{O}^{2} \cdot R ) ρ = ( 2 π R l {R}_{O}^{2} R ) ρ = ( 2 π l {R}_{O}^{4} ) ρ . \qquad Equation 45$$

If Ro is greater than R then, by offsetting the vibration of the cylindrical element from the axis, the loading factors are amplified the square of the ratio between Ro and R, i.e. an amplification of (R_{O}/R)². The loading factors are amplified by (R_{O}/R)⁴ in the case of J_{F}, i.e. the fourth power of the ratio between R_{O} and R.

However, these equations only hold under geometric damping of the cylindrical element. By offsetting the cylindrical element, it no longer undergoes pure torsional oscillation but instead vibrates laterally at the displaced distance. These loading factor equations do not automatically apply because, under lateral vibration, the cylindrical element may form a dipole wave field rather than a monopole wave field.

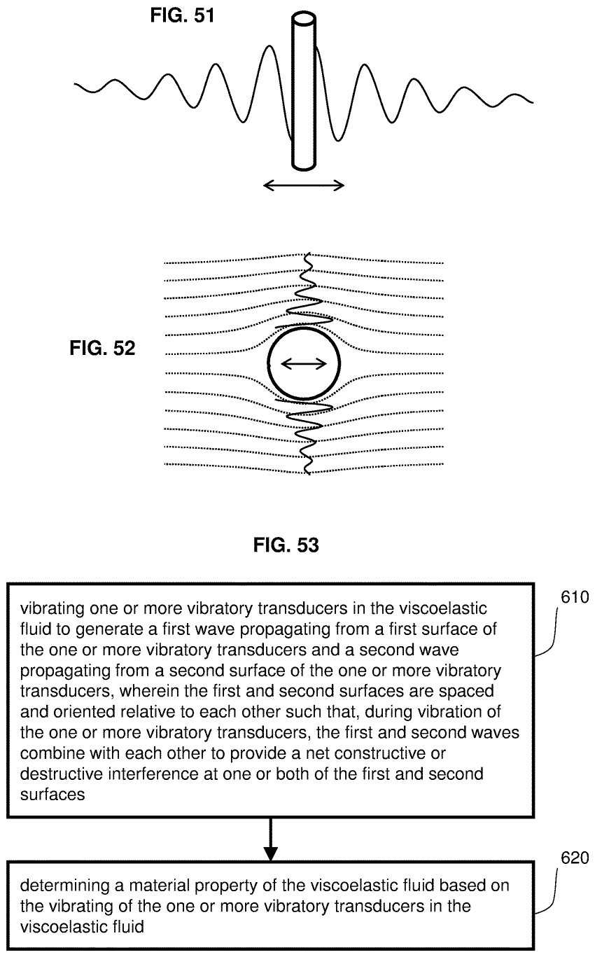

FIG. 51 illustrates a cylindrical element under lateral vibration causing a dipole wave field. Under a dipole wave field, there is a 180° phase difference between the wave field on each side of the cylindrical element. The formation of a dipole wave field is problematic because the resulting waves are pressure (P) waves rather than shear(S) waves. The fluid mechanics of pressure waves are different from shear waves and the previously defined relationships for shear waves no longer apply.

For example, a damping factor is defined differently for a shear wave than for a pressure wave. For a shear wave, the shear rate and the stresses created by the shear rate lead to a relatively well-defined and controllable damping wave. By contrast, pressure waves follow what is known as ‘quadratic damping’, where the damping force is proportional to the square of the velocity. This leads to a damping factor of the form:

$${C}_{quadratic} = b \cdot v \qquad Equation 46$$

where b is a constant and v is the velocity. In other words, the damping factor unhelpfully varies with the vibrational velocity.

However, a monopole wave field may be maintained if the Reynolds number is kept low. The Reynolds number signifies a ratio between inertial forces and viscous forces. As the Reynolds number reduces, viscous forces become larger relative to inertial forces.

FIG. 52 illustrates laminar flow around a cylinder vibrating in a left-right direction perpendicular to the cylinder's axis. With laminar flow, the forces across either side of the cylinder become shear and so shear waves propagate as a result of lateral vibration. Without wishing to be bound by theory, it is believed that laminar flow leads to inertial forces being sufficiently low relative to viscous forces that the wave field is largely or at least partially defined by shear waves generated from the upper and lower portions of the cylinder cross-section, i.e. perpendicular to the axis and the direction of vibration. As a result, as the Reynolds number is reduced, the degree to which the wave field has a dipole form is reduced and the degree to which the wave field has a monopole form is increased. The low Reynolds number has restored a partial shear wave field which is in phase through out its propagation space. The benefits of geometric damping are retained but with the advantage of increased loading factor gain from offsetting the element from the axis.

Advantageously, the cylindrical element displaced from the axis may achieve a relatively high degree of amplification with little increase in size and weight because a relatively low Reynolds number is readily achievable with small length scales. In some implementations of the techniques of this disclosure, fluid loading factor may be achieved that are equivalent to those of much larger and heavier vibratory elements.

It is further recognised that a relatively low Reynolds number is readily achievable for micro- and nanoscale devices for almost all fluids of interest, regardless of their physical properties. Sub-micron needle structures on a vibrating substrate may form the same radially displaced elements discussed above and achieve the same benefits of geometric damping. Some implementations may feature multiple cylindrical elements or cylinder-like elements, such as pins and spikes, formed by micro- or nano-manufacturing processes, may allow miniature surfaces to present high fluid load factors.

It is further recognised that a low Reynolds number may lead to a wave field is that is only partially defined by shear waves and so expressions for the loading factor under geometric damping conditions might not be identical to the expressions set out above, but may be proportional to those expressions, the constant of proportionality depending on the degree to which the wave field is defined by shear waves. In the following expressions, a constant of proportionality, h, is introduced, wherein h would have a value of 1 if the wave field is wholly defined by shear waves, and would have a value of 0.5 if the wave field is 50% defined by shear waves, which may be a reasonable assumption in practice:

$${C}_{F} = h ( 2 π l {R}_{O}^{2} ) {μ}^{′} \qquad Equation 47$$

$${K}_{F} = h ( 2 π l {R}_{O}^{2} ) {G}^{′} \qquad Equation 48$$

$${J}_{F} = h ( 2 π l {R}_{O}^{4} ) ρ . \qquad Equation 49$$

If h is assumed to be 0.5, then the above expressions simplify to:

$${C}_{F} = ( π l {R}_{O}^{2} ) {μ}^{′} \qquad Equation 50$$

$${K}_{F} = ( π l {R}_{O}^{2} ) {G}^{′} \qquad Equation 51$$

$${J}_{F} = ( π l {R}_{O}^{4} ) ρ . \qquad Equation 52$$

The meaning of a ‘low’ Reynolds number in the context of the present disclosure is that the Reynolds number is sufficiently low that laminar flow is obtained and the flow due to the vibration may be characterised to some degree by a shear wave field and the advantages of geometric damping provided to at least some degree. It is recognised that a laminar-to-turbulent flow transition occurs over a range of Reynolds number values and the precise range over which this transition takes place is dependent on geometry. A lower Reynolds number is more likely to result in flow behaviour leading to a partial shear wave field than a higher Reynolds number. Without wishing to be bound by theory, it is believed that the degree to which a shear wave field develops, and thus some advantages of the techniques of this disclosure are obtained, is dependent on the Reynolds number. For example, a Reynolds number of 1000 may exhibit a degree of laminar-like flow and result in a shear wave field to some degree. A Reynolds number of 100 may exhibit an even greater degree of laminar-like flow and result in a shear wave field to an even greater degree. A Reynolds number of 10 may exhibit an even greater degree of laminar-like flow and result in a shear wave field to an even greater degree. A Reynolds number of 1 may exhibit an even greater degree of laminar-like flow and result in a shear wave field to an even greater degree. In general, a lower Reynolds number may be preferred but the reader will recognise that achieving a lowest possible Reynolds number has to be balanced with other technical considerations.

Provided geometric damping conditions are satisfied, the wave propagation depth is determined by the geometry of the cylindrical element, and specifically its radius.

In view of the above discussion of geometric damping, a radiator or detector surface may be a surface of or comprise an elongate member. Such elongate members may be characterised by a width, a half width that is equal to half of the width, and a length that is greater than the width, wherein the half width is less than a propagation depth of a shear wave in the fluid at the vibration frequency, more preferably less than 50% of the propagation depth. Such elongate members may be non-colinear or offset from an axis of vibration such as an axis of torsional vibration. During vibration, the flow of fluid around the elongate member may be laminar flow.

In some embodiments, the half width of the elongate member is less than 75% of the propagation depth, optionally less than 60%, optionally less than 50%, optionally less than 40%, optionally less than 25%, optionally less than 10%, optionally less than 5%, optionally less than 2%, optionally less than 1%, or optionally less than 0.5%.

In some embodiments, the elongate member has a substantially or wholly circular cross section along 50%, 70%, 90% or 100% of its length. Optionally, along 50%, 70%, 90% or 100% of its length, the elongate member has a cross section that has a circularity in the range 0.75 to 1, optionally in the range 0.8 to 1, optionally in the range 0.85 to 1, optionally in the range 0.9 to 1, optionally in the range 0.95 to 1, more preferably in the range 0.9 to 1, optionally in the range 0.95 to 1, wherein the circularity of a cross section shape is calculated by 4πA/p², where A is the convex area of the cross section shape and p is the convex perimeter of the cross section shape.

In some embodiments, a half width of the elongate member calculated at a point along its length is based on the convex perimeter or the convex area of the cross section of the elongate member at that point along its length. Optionally, the half width is calculated based on the convex perimeter of the shape of the cross section by the expression p/2π. Alternatively, the half width may be calculated based on the convex area of the shape of the cross section by the expression √(A/π). If the elongate member has a circular cross section then both of these expressions produce the radius of the circle and so the half width of a circular cross section is the radius of the circle.

In some embodiments, the elongate member has a constant cross section along more than 50%, more than 60%, more than 70%, more than 80%, more than 90% or 100% of its length.

In some embodiments, the elongate member only has a constant cross section along less than 50%, less than 40%, less than 30%, less than 20%, less than 10% of its length, or the cross section varies continuously along its length.

In some embodiments, the area of the cross section increases or decreases monotonically along the length of the elongate member.

In some embodiments, the elongate member is straight.

In some embodiments, the elongate member is axially symmetric along its length.

In some embodiments, the elongate member is non-straight. For example, the elongate member may comprise a closed loop.

In some embodiments, the elongate member comprises one of: a circular cylinder, a cone, a frustrum of a cone, a torus, and an arcuate portion of a torus.

In some embodiments, the half width the elongate member that is less than the propagation depth is a maximum half width along the length of the elongate member.

In some embodiments, the half width of the elongate member that is less than the propagation depth is an average half width along the length of the elongate member. Optionally, the average half width is calculated as an arithmetic mean of the half width along the length of the elongate member or is an average half width that is calculated as twice a volume of the elongate member divided by the surface area of the elongate member.

In some embodiments, the width of the elongate member is greater than 0.5 mm, and/or greater than 1 mm, and/or greater than 2 mm, and/or greater than 5 mm, and/or greater than 10 mm, and/or greater than 20 mm, and/or greater than 50 mm.

In some embodiments, the length of the elongate member is greater than a multiple of the half width of the elongate member (the half width being half of the width of the elongate member), and wherein the multiple is one of: 2, 3, 4, 5, 6, 7, 8, 9, 10, 11, 12, 13, 14, 15, 16, 17, 18, 19, 20, 25, 30, 35, 40, 45, or 50. Expressed another way, the length of the elongate member may be greater than a multiple of the width of the elongate member, wherein the multiple is one of 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, and 25.

In some embodiments, the width of the elongate member is between 1 nm and 500 nm. Such embodiments may be described as ‘nanoscale’ or nanoscopic-scale embodiments. In some other embodiments, the width of the elongate member is between 500 nm and 500 μm. Such embodiments may be described as ‘microscale’ or microscopic-scale embodiments. An appropriately dimensioned device, which may be a nanoscale or microscale device, may vibrate at a low frequency to advantageously measure fluid properties of fluids with low viscosities, such as less than 1 mPa·s, or may vibrate at a high frequency to advantageously measure fluid properties of fluids with low viscosities, such as less than 1 mPa·s because, at such small scales, the width of the elongate member may still be small relative to the propagation depth at such high frequencies.

In some embodiments, a vibratory transducer element comprises a shaft that has a longitudinal axis, wherein the elongate member is connected to the shaft and wherein the elongate member is not collinear with the longitudinal axis of the shaft. Optionally, during vibration of the vibratory transducer element at the vibration frequency, the flow of fluid around the elongate member is laminar flow. Alternatively or additionally, a Reynolds number, Re, of fluid flow around the elongate member is less than one, wherein the Reynolds number is equal to 2Rvρ/μ, where μ is a viscosity of the fluid, ρ is a density of the fluid, R is the half width of the elongate member, and v is a maximum (vibrational) velocity of the elongate member relative to the fluid during vibration of the vibratory transducer, wherein optionally the Reynolds number is less than 1000, or less than 300, or less than 100, or less than 30, or less than 10, or less than 3, or less than 1, or less than 0.9, or less than 0.8, or less than 0.75, or less than 0.7, or less than 0.6, or less than 0.5, or less than 0.4, or less than 0.3, or less than 0.25, or less than 0.2, or less than 0.1. Alternatively or additionally, the elongate member may have a first end and a second end, wherein one or both of the first and second end is spaced from the longitudinal axis of the shaft by an offset distance that is greater than the half width of the elongate member, wherein, optionally, the offset distance is greater than a multiple of the width and the multiple is 2, 3, 4, 5, 6, 7, 8, 9, 10, 11, 12, 13, 14, 15, 20, 30 or 50. Alternatively or additionally, the vibratory transducer element may comprise a plurality of elongate members connected to the shaft that are each not collinear with the axis of the shaft, each having a half width that is less than the propagation depth of a shear wave in the fluid at the vibration frequency, and wherein, optionally, the half width of a first elongate member of the plurality of elongate members is different from the half width of a second elongate member of the plurality of elongate members, and wherein, optionally, two, three, four, five or more of the elongate members of the plurality of elongate members may have uniquely different half widths. Alternatively or additionally, the elongate member may comprise a first end and a second end, wherein the elongate member is connected to the shaft at the first end and optionally also at the second end. Alternatively or additionally, the shaft may comprise a bob and the elongate member may be connected to the shaft at the bob.

Vibratory transducers in accordance with the techniques described herein may be used to determine a physical property of a fluid by vibrating the vibrating transducer in a fluid at a vibration frequency and determining a quantity indicative of a degree of damping based on the vibration. For example, to make a measurement of the viscosity, the Q factor of the vibration can be determined. The Q factor is a dimensionless parameter that indicates the level of damping of a resonator, wherein the level of damping is a function of the viscosity. In particular, it indicates the degree to which a resonator is underdamped. On a plot of frequency response, a high Q factor provides a high and narrow peak at the resonant frequency whereas a low Q factor provides a low and wide peak. Due to the change in width of the peak with damping, the Q factor can be defined as the ratio of the resonant frequency to the resonant bandwidth:

$$Q = \frac{{ω}_{R}}{Δ ω} \qquad Equation 53$$

wherein ω_{R} is the resonant frequency in radians per second and Δω is the Full Width at Half Maximum (FWHM), the bandwidth over which the power of the vibration is greater than half of the maximum (or equivalently the amplitude of vibration is greater than the maximum amplitude at resonance divided by √2), i.e. the bandwidth between the 3 dB points. The fluid viscosity is a function of the Q factor.

It should be noted that the measurement of viscosity at or corresponding to a frequency of vibration may comprise making amplitude measurements at more than one frequency to estimate the Q factor but a single viscosity measurement is obtained at a frequency corresponding to the or a resonant frequency. For example, the bandwidth can be determined based on the frequencies required to cause the amplitude to drop to a factor of 1/√2 of the maximum amplitude at resonance. As a non-limiting example, the frequencies required to cause the amplitude to drop to a factor of 1/√2 of the maximum amplitude at resonance may be determined by performing a frequency sweep around the resonant frequency, but the skilled reader will recognize that the 3 dB point frequencies can be identified by various other techniques.

Another approach to determining the Q factor is to measure the amplitude of vibration at a series of frequencies around the resonant frequency and fit a parabola by the method of least squares to the frequency and amplitude values (or logarithms thereof). The 3 dB points can then be obtained as solutions to a quadratic equation based on the parabola of best fit to the measurements.

Another approach to determining the Q factor is by logarithmic decrement. By ceasing to drive the transducer and measuring the decay of vibrations, the Q factor may be determined by monitoring time series of the vibrations and determining the natural logarithm of the ratio of two successive peaks, A₁ and A₂, by the following expression:

$$Q = \frac{1}{2} \sqrt{1 + {( \frac{2 π}{ln ( \frac{{A}_{1}}{{A}_{2}} )} )}^{2}} . \qquad Equation 54$$

As discussed above, the loss factor is the inverse of the Q factor and so can be readily determined based on the above-described approaches.

FIG. 53 illustrates a flow chart of a method in accordance with the techniques of this disclosure. The method includes a first step 610 of vibrating one or more vibratory transducers in the viscoelastic fluid to generate a first wave propagating from a first surface of the one or more vibratory transducers and a second wave propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces.

The method includes a second step 620 of determining a material property of the viscoelastic fluid based on the vibrating of the one or more vibratory transducers in the viscoelastic fluid.

In accordance with the techniques of this disclosure, a means for vibrating one or more vibratory transducers may comprise an electronic device configured to provide a control signal to one or more vibratory transducers to cause the one or more vibratory transducers to vibrate in a fluid in a manner in accordance with the techniques of this disclosure.

In accordance with the techniques of this disclosure, a means for determining a material property of the viscoelastic fluid based on the vibrating of the one or more vibratory transducers in the viscoelastic fluid may comprise an electronic device configured to record measurements of the vibrating of the one or more vibratory transducers and process the measurements to determine a material property.

The means for vibrating the one or more vibratory transducers and the means for determining the material property of the viscoelastic fluid based on the one or more vibratory transducers may be the same electronic device (i.e. a single electronic device causes the vibration and determines the material property) or may be different electronic devices.

The skilled reader will further appreciate that the various illustrative logical blocks, configurations, modules, circuits, and algorithm steps described in connection with the embodiments disclosed herein may be implemented as electronic hardware, computer software, or combinations of both. To clearly illustrate this interchangeability of hardware and software, various illustrative components, blocks, configurations, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The steps of a method or algorithm described in connection with the embodiments disclosed herein may be embodied directly in hardware, in a software module executed by a processor, or in a combination of the two. A software module may reside in random access memory (RAM), flash memory, read-only memory (ROM), programmable read-only memory (PROM), erasable programmable read-only memory (EPROM), electrically erasable programmable read-only memory (EEPROM), registers, hard disk, a removable disk, a compact disc read-only memory (CD-ROM), or any other form of storage medium known in the art. An exemplary storage medium is coupled to the processor such that the processor can read information from, and write information to, the storage medium. In the alternative, the storage medium may be integral to the processor. The processor and the storage medium may reside in an application-specific integrated circuit (ASIC). The ASIC may reside in a computing device or a user terminal. In the alternative, the processor and the storage medium may reside as discrete components in a computing device or user terminal.

Material in accordance with this disclosure may be described by way of the following numbered aspects:

Aspect 1. A method of measuring a material property of a viscoelastic fluid using one or more vibratory transducers, the method comprising:
vibrating one or more vibratory transducers in the viscoelastic fluid to generate a first wave propagating from a first surface of the one or more vibratory transducers and a second wave propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces; and
determining a material property of the viscoelastic fluid based on the vibrating of the one or more vibratory transducers in the viscoelastic fluid.

Aspect 2. The method of aspect 1, wherein the first and second waves combine with each other to provide a net destructive interference.

Aspect 3. The method of aspect 1, wherein the first and second waves combine with each other to provide a net constructive interference.

Aspect 4. The method of aspect 1 or aspect 2, wherein the first and second waves are shear waves.

Aspect 5. The method of any of aspects 1 to 4, wherein determining a measurement of a material property of the viscoelastic fluid comprises determining a loss factor or a Q factor of the vibration of the one or more vibratory transducers in the viscoelastic fluid.

Aspect 6. The method of aspect 5, wherein the determined loss factor or Q factor is a monotonic function of the viscosity or storage modulus of the viscoelastic fluid.

Aspect 7. The method of any pf aspects 1 to 6, wherein tan A of the viscoelastic fluid is less than 1, wherein tan A is the loss tangent of the viscoelastic fluid.

Aspect 8. The method of any of aspects 1 to 7, wherein one or both of the first and second surfaces is curved.

Aspect 9. The method of aspect 8, wherein one or both of the first and second surfaces is convex.

Aspect 10. The method of aspect 8 or aspect 9, wherein one or both of the first and second surfaces is partially or wholly concave.

Aspect 11. The method of aspect 10, wherein the first surface is partially or wholly concave and the first wave generated from the first surface is configured to focus at a focal distance from the first surface, wherein: i) the second surface is located further from the first surface than the focal distance is from the first surface, or ii) the second surface is located nearer to the first surface than the focal distance is from the first surface, or iii) the second surface is located at the focal distance from the first surface.

Aspect 12. The method of any of aspects 1 to 11, wherein the first surface comprises a concave portion configured to focus the first wave, wherein the concave portion comprises a first region of the concave portion and a second region of the concave portion that is configured to vibrate out of phase with the first region of the concave portion to generate a wave that is out of phase with a wave generated from the first region of the concave portion.

Aspect 13. The method of aspect 12, wherein vibrating the one or more vibratory transducers comprises torsionally vibrating the concave portion of the first surface about a vibrational axis that extends through the concave portion of the first surface, wherein: i) the first region of the concave portion and second region of the concave portion are located on opposing sides of the vibrational axis, and/or ii) the concave portion of the first surface is axially symmetric about the vibrational axis, and/or iii) the vibrational axis extends through a focused region of the concave portion.

Aspect 14. The method of aspect 12 or aspect 13, wherein the depth profile of the concave portion in a lateral cross section through the vibrational axis is smooth and preferably has the shape of a circular, parabolic or elliptical arc.

Aspect 15. The method of aspect 12, wherein vibrating the one or more vibratory transducers comprises vibrating the first and second regions of the concave portion in a path, such as a linear path or a circular path.

Aspect 16. The method of aspect 15, wherein the first region of the concave portion and the second region of the concave portion have constant concave depth profiles in the direction of the path.

Aspect 17. The method of aspect 16, wherein vibrating the one or more vibratory transducers comprises vibrating the first and second regions of the concave portion in the direction of the path.

Aspect 18. The method of aspect 16 or aspect 17 aspect, wherein the depth profiles of the first and second regions of the concave portion are smooth and preferably have the shape of a circular, parabolic or elliptical arc.

Aspect 19. The method of any of aspects 1 to 18, wherein one or both of the first and second surfaces comprise an elongate member.

Aspect 20. The method of aspect 19, wherein vibrating the one or more vibratory transducers in the viscoelastic fluid comprises vibrating the one or more vibratory transducers through or about a respective vibrational axis of the one or more vibratory transducers, wherein the vibrational axis is not colinear with at least one elongate member of corresponding to the first surface or second surface.

Aspect 21. The method of aspect 19 or aspect 20, wherein vibrating the one or more vibratory transducers in the viscoelastic fluid comprises vibrating the one or more vibratory transducers torsionally about a common vibrational axis.

Aspect 22. The method of aspect 21, wherein one or both of the first and second surfaces comprise an elongate member configured in the form of a ring.

Aspect 23. The method of aspect 22, wherein an axis through the centre of the ring is colinear with the common vibrational axis.

Aspect 24. The method of aspect 22 or aspect 23, wherein one or both of the first and second surfaces is concave and configured to focus waves to the ring.

Aspect 25. The method of any of aspects 21 to 24, wherein the first surface comprises an elongate member configured in the form of a ring that is connected to the second surface by one or more support members that offset the first surface from the second surface.

Aspect 26. The method of aspect 25, wherein the second surface comprises a concave portion configured to generate shear waves under torsional vibration about the common vibrational axis, the generated shear waves focusing toward the first surface.

Aspect 27. The method of aspect 19 or aspect 20, wherein the one or more vibratory transducers comprise a shaft having a longitudinal axis extending along the shaft and a plurality of elongate members extending outward from the longitudinal axis and spaced from each other, wherein vibrating the one or more vibratory transducers comprises torsionally vibrating the shaft about the longitudinal axis.

Aspect 28. The method of aspect 27, wherein the plurality of elongate members are connected to the shaft.

Aspect 28A. The method of aspect 28, wherein the plurality of elongate members extend outward from the shaft in a wholly radial direction from the longitudinal axis.

Aspect 29. The method of aspect 27, wherein the shaft comprises a bob and the plurality of elongate members are connected to the shaft at the bob.

Aspect 29A. The method of aspect 29, wherein the plurality of elongate members extend outward from the bob in a wholly radial direction from the longitudinal axis.

Aspect 29B. The method of aspect 19 or aspect 20, wherein the one or more vibratory transducers comprise a shaft having a longitudinal axis extending along the shaft and a plurality of elongate members extending in a direction aligned with the longitudinal axis and spaced from each other, wherein vibrating the one or more vibratory transducers comprises torsionally vibrating the shaft about the longitudinal axis.

Aspect 29C. The method of aspect 29B, wherein none of the plurality of elongate members are colinear with the longitudinal axis of the shaft.

Aspect 29D. The method of aspect 29B or aspect 29C, wherein some or all of the plurality of elongate members extend axially outward from an end of the shaft.

Aspect 29E. The method of aspect 29B or aspect 29C, wherein the shaft comprises a bob and the plurality of elongate members are connected to the shaft at the bob.

Aspect 29F. The method of aspect 29E, wherein the bob is cylindrical and coaxial with the longitudinal axis.

Aspect 29G. The method of aspect 29E or 29F, wherein the plurality of elongate members extend axially outward from an end of the bob.

Aspect 29H. The method of any of aspects 29B to 29G, wherein the plurality of elongate members are distributed evenly around the longitudinal axis of the shaft.

Aspect 29I. The method of any of aspects 29B to 29H, wherein the plurality of elongate members are each positioned at a same radial distance from the longitudinal axis of the shaft.

Aspect 30. The method of any of aspects 25 to 29I, wherein the first and second waves are shear waves and wherein one or both of the first and second surfaces comprise an elongate member characterised by a width, a half width that is equal to half of the width, and a length that is greater than the width, wherein the half width is less than a propagation depth of a shear wave in the fluid at the vibration frequency.

Aspect 31. The method of aspect 30, wherein the propagation depth is a distance over which an amplitude of a shear wave propagating in the fluid at the vibration frequency is reduced by a factor of 1/e, wherein e is the base of natural logarithms.

Aspect 32. The method of aspect 30 or aspect 31, wherein the propagation depth of a shear wave propagating in the fluid at the vibration frequency is given by the expression:

$$\frac{1}{sin \frac{Δ}{2}} \sqrt{\frac{μ}{ρ ω sin Δ} ,}$$

wherein μ is a viscosity of the fluid, ρ is a density of the fluid, ω is the angular frequency of vibration, and Δ varies between 0 and π/2 (in radians) and is defined by the loss tangent, tan Δ, and wherein tan Δ is equal to the following expression, in which G′ is a storage modulus of the fluid:

$$\frac{μ ω}{{G}^{′}} .$$

Aspect 33. The method of any of aspects 30 to 32, wherein the half width is less than 50% of the propagation depth.

Aspect 34. The method of any of aspects 30 to 33, wherein the one or more vibratory transducers comprise a shaft that has a longitudinal axis, wherein an elongate member is connected to the shaft and wherein the elongate member is not collinear with the longitudinal axis of the shaft.

Aspect 35. The method of any of aspects 30 to 34, wherein the shaft comprises a bob and the elongate member is connected to the shaft at the bob.

Aspect 36. The method of any of aspects 30 to 36, wherein the vibratory transducer element comprises a plurality of elongate members connected to the shaft that are each not collinear with the longitudinal axis of the shaft, each having a half width that is less than the propagation depth of a shear wave in the fluid at the vibration frequency.

Aspect 37. The method of aspect 36, wherein the half width of a first elongate member of the plurality of elongate members is different from the half width of a second elongate member of the plurality of elongate members.

Aspect 38. The method of any of aspects 30 to 37, wherein, along at least 50% of its length, the elongate member has a cross section that has a circularity in the range 0.75 to 1, wherein the circularity of a cross section is calculated by:

$$\frac{4 π A}{{p}^{2}} ,$$

where A is the convex area of the cross section and p is the convex perimeter of the cross section.

Aspect 39. The method of any of aspects 30 to 38, wherein the elongate member has a constant cross section along at least 90% of its length or has a constant cross section along no more than 10% of its length.

Aspect 40. The method of any of aspects 30 to 39, wherein the elongate member is straight or non-straight or comprises one of: a circular cylinder, a cone, a frustrum of a cone, a torus, and an arcuate portion of a torus.

Aspect 41. The method of any of aspects 30 to 40, wherein vibrating the vibratory transducer comprises vibrating the vibratory transducer with an oscillatory rotational motion and/or an oscillatory rectilinear motion and/or an oscillatory curvilinear motion.

Aspect 42. The method of aspect 41, wherein the elongate member is straight and wherein vibrating the vibratory transducer comprises vibrating the elongate member with an oscillatory rotational motion about an axis along the length of the elongate member.

Aspect 43. The method of aspects 30 to 42, wherein the length of the elongate member is greater than twice the width of the elongate member.

Aspect 44. The method of any of aspects 30 to 43, wherein the half width of the elongate member is greater than 0.5 mm, wherein a viscosity of the fluid is greater than 100 Pa·s, wherein a density of the fluid is between 500 kg/m³ and 1500 kg/m³, and wherein the frequency of vibration is less than 10 kHz.

Aspect 45. The method of any of aspects 35 to 44, wherein the flow of fluid around the elongate member is laminar flow during vibration of the elongate member at the vibration frequency.

Aspect 46. The method of any of aspects 35 to 45, wherein the elongate member has a first end and a second end, wherein one or both of the first and second ends is spaced from the longitudinal axis of the shaft by an offset distance that is greater than the half width of the elongate member.

Aspect 47. The method of any of aspects 35 to 46, wherein, during vibration of the one or more vibratory transducers in the fluid at a vibration frequency, a Reynolds number, Re, of fluid flow around the elongate member is less than 1000, preferably less than 100, more preferably less than 10, even more preferably less than 1, wherein the Reynolds number is given by

$$Re = \frac{2 R v ρ}{{μ}^{′}} ,$$

wherein μ′ is a viscosity of the fluid, p is a density of the fluid, R is the half width of the elongate member, and v is a maximum velocity of the elongate member relative to the fluid during vibration of the one or more vibratory transducers.

Aspect 48. The method of any of aspects 30 to 34, wherein the elongate member is ring-shaped.

Aspect 49. The method of any of aspects 1 to 48, wherein vibrating the one or more vibratory transducers comprises vibrating the one or more vibratory transducers at a vibration frequency, wherein the vibration frequency is between 100 Hz and 100 kHz, preferably between 200 Hz and 10 kHz, more preferably between 300 Hz and 5 kHz, and more preferably between 500 Hz and 2 kHz, even more preferably between 700 Hz and 1300 Hz, even more preferably at or approximately at 1 kHz, such as within 100 Hz of 1 kHz, preferably within 50 Hz of 1 kHz.

Aspect 50. The method of any of aspects 1 to 49, wherein the one or more vibratory transducers vibrate torsionally about a common axis.

Aspect 51. The method of any of aspects 1 to 50 wherein determining the material property of the viscoelastic fluid based on the measurement of vibration comprises determining one or more of: a viscosity, a viscoelasticity, a density, a fluid stiffness, a loss tangent, a storage modulus, a loss modulus, and a yield stress.

Aspect 52. The method of any of aspects 1 to 51, wherein determining the material property of the viscoelastic fluid based on the vibrating of the one or more vibratory transducers in the viscoelastic fluid comprises determining a quantity indicative of a degree of damping in the fluid at the vibration frequency.

Aspect 53. The method of aspect 52, wherein determining a quantity indicative of a degree of damping of vibration in the fluid at the vibration frequency comprises determining a loss factor or a Q factor.

Aspect 54. The method of aspect 52 or aspect 53, wherein determining a quantity indicative of a degree of damping in the fluid at the vibration frequency comprises determining a first quantity indicative of a degree of damping at the vibration frequency, wherein the method further comprising vibrating the vibratory transducer at a further vibration frequency and determining a second quantity indicative of a degree of damping at the further vibration frequency, wherein determining the material property of the fluid comprises determining a viscoelasticity of the fluid based on the quantities indicative of the degree of damping at the vibration frequency and at the further vibration frequency.

Aspect 55. The method of aspect 52 or aspect 53, wherein determining the material property of the fluid comprises determining a density of the fluid based on a measurement of a resonant frequency of the vibratory transducer in the fluid.

Aspect 56. An apparatus for measuring a material property of a viscoelastic fluid using one or more vibratory transducers, the apparatus comprising:
one or more vibratory transducers, the one or more vibratory transducers comprising a first surface and a second surface;
means for vibrating the one or more vibratory transducers such that, when vibrated in a viscoelastic fluid, a first wave is generated propagating from a first surface of the one or more vibratory transducers and a second wave is generated propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces; and
means for determining a material property of the viscoelastic fluid based on the vibrating of the one or more vibratory transducers in the viscoelastic fluid including the net constructive or destructive interference.

Aspect 57. The apparatus of aspect 56, wherein the first surface comprises a concave portion.

Aspect 58. The apparatus of aspect 56 or aspect 57, wherein the first surface comprises an elongate member.

Aspect 59. The apparatus of any of aspects 56 to 58, wherein the second surface comprises an elongate member.

Aspect 60. The apparatus of any of aspects 56 to 58, wherein the second surface is flat, wherein optionally the first surface comprises an elongate member extending outward from the second surface.

Aspect 61. The apparatus of aspect 60, wherein the first surface comprises an elongate member extending in a direction normal to the second surface.

Aspect 62. The apparatus of aspect 61, wherein the first surface comprises an elongate member configured in the form of a ring.

Aspect 63. The apparatus of aspect 58, wherein the second surface faces radially outward from an axis of torsional vibration of the vibratory transducer on which the second surface is located and is configured to generate a shear wave extending radially outward from the axis of torsional vibration, wherein optionally the first surface comprises an elongate member that extends outward from the second surface.

Aspect 64. The apparatus of aspect 64, wherein the first surface comprises an elongate member that extends in a direction normal to the second surface.

Aspect 65. The apparatus of aspect 66, wherein the first surface comprises an elongate member that extends circumferentially around the second surface or in a direction aligned with the axis of torsional vibration.

Aspect 66. The apparatus of any of aspects 60 to 65, wherein the second surface comprises a concave portion configured to generate a wave in the fluid that focuses at a focal distance from the second surface, wherein: i) the first surface is located further from the second surface than the focal distance from the second surface, or ii) the first surface is located nearer to the second surface than the focal distance from the second surface, or iii) the first surface is located at the focal distance from the second surface.

Aspect 67. The apparatus of any of aspects 56 to 71, wherein the first surface comprises an elongate member configured in the form of a ring that is connected to the second surface by one or more support members that offset the first surface from the second surface.

Aspect 68. The apparatus of aspect 67, wherein the second surface comprises a concave portion configured to generate shear waves under torsional vibration about the common vibrational axis, the generated shear waves focusing toward the first surface.

Aspect 69. The apparatus of any of aspects 56 to 66, wherein the one or more vibratory transducers comprise a shaft having a longitudinal axis extending along the shaft and a plurality of elongate members extending outward from the longitudinal axis and spaced from each other, wherein vibrating the one or more vibratory transducers comprises torsionally vibrating the shaft about the longitudinal axis.

Aspect 70. The apparatus of aspect 69, wherein the plurality of elongate members are connected to the shaft and extend radially outward from the shaft.

Aspect 70A. The apparatus of aspect 70, wherein the plurality of elongate members extend outward from the shaft in a wholly radial direction from the longitudinal axis.

Aspect 71. The apparatus of aspect 69, wherein the shaft comprises a bob and the plurality of elongate members are connected to the shaft at the bob.

Aspect 71A. The apparatus of aspect 71, wherein the plurality of elongate members extend outward from the bob in a wholly radial direction from the longitudinal axis.

Aspect 71B. The apparatus of any of aspects 56 to 66, wherein the one or more vibratory transducers comprise a shaft having a longitudinal axis extending along the shaft and a plurality of elongate members extending in a direction aligned with the longitudinal axis and spaced from each other, wherein vibrating the one or more vibratory transducers comprises torsionally vibrating the shaft about the longitudinal axis.

Aspect 71C. The apparatus of aspect 71B, wherein none of the plurality of elongate members are colinear with the longitudinal axis of the shaft.

Aspect 71D. The apparatus of aspect 71B or aspect 71C, wherein some or all of the plurality of elongate members extend axially outward from an end of the shaft.

Aspect 71E. The apparatus of aspect 71B or aspect 71C, wherein the shaft comprises a bob and the plurality of elongate members are connected to the shaft at the bob.

Aspect 71F. The apparatus of aspect 71E, wherein the bob is cylindrical and coaxial with the longitudinal axis.

Aspect 71G. The apparatus of aspect 71E or 71F, wherein the plurality of elongate members extend axially outward from an end of the bob.

Aspect 71H. The apparatus of any of aspects 71B to 71G, wherein the plurality of elongate members are distributed evenly around the longitudinal axis of the shaft.

Aspect 711. The apparatus of any of aspects 71B to 71H, wherein the plurality of elongate members are each positioned at a same radial distance from the longitudinal axis of the shaft.

Aspect 72. The apparatus of any of aspects 56 to 711, wherein one or both of the first and second surfaces comprise an elongate member characterised by a width, a half width that is equal to half of the width, and a length that is greater than the width, wherein the half width is less than a propagation depth of a shear wave in the fluid at the vibration frequency.

Aspect 73. The apparatus of aspect 72, wherein the half width is less than 50% of the propagation depth.

Aspect 74. The apparatus of aspect 72 or aspect 73, wherein the one or more vibratory transducers comprise a shaft that has a longitudinal axis, wherein an elongate member is connected to the shaft and wherein the elongate member is not collinear with the longitudinal axis of the shaft.

Aspect 75. The apparatus of aspect 74, wherein, during vibration of the one or more vibratory transducers in the fluid at a vibration frequency, a Reynolds number, Re, of fluid flow around the elongate member is less than 1000, preferably less than 100, more preferably less than 10, even more preferably less than 1, wherein the Reynolds number is given by

$$Re = \frac{2 R v ρ}{{μ}^{′}} ,$$

wherein μ′ is a viscosity of the fluid, ρ is a density of the fluid, R is the half width of the elongate member, and v is a maximum velocity of the elongate member relative to the fluid during vibration of the vibratory transducer.

Aspect 76. The apparatus of any of aspects aspect 56 to 75, wherein the first surface is flat and/or comprises a concave portion configured to generate a wave in the fluid that focuses at a focal distance from the first surface.

Aspect 77. The apparatus of aspect 76, wherein the first surface comprises a concave portion configured to generate a wave in the fluid that focuses at a focal distance from the first surface.

Aspect 78. The apparatus of aspect 77, wherein the second surface is located further from the first surface than the focal distance is from the first surface.

Aspect 79. The apparatus of aspect 77, wherein the second surface is located nearer to the first surface than the focal distance is from the first surface.

Aspect 80. The apparatus of any of aspects 76 to 79, wherein the second surface is flat and/or the first and second surfaces each comprise a concave portion configured to generate a wave in the fluid that focuses at a focal region between the first and second surfaces.

Aspect 81. The apparatus of aspect 80, wherein the first and second surfaces each comprise a concave portion configured to generate a wave in the fluid that focuses at a focal region between the first and second surfaces.

Aspect 82. The apparatus of aspect 81, wherein the focal region is equidistant between the first and second surfaces.

Aspect 83. The apparatus of aspect 81, wherein the first and second surfaces are configured to vibrate torsionally about a common axis and the concave portions of each of the first and second surfaces are axially symmetric about the common axis of vibration.

Aspect 84. The apparatus of aspect 83, wherein one or both of the concave portions of the first and second surfaces are circular grooves in the first and second surfaces circumscribing the common axis.

Aspect 85. The apparatus of any of aspects 76 to 84, wherein first surface is configured to vibrate torsionally about an axis normal to the first surface, and wherein the second surface circumscribes the axis and faces radially outward from the axis of vibration, wherein the radius of the second surface is less than a maximum outermost extent of the first surface about the axis, and wherein the second surface is axially displaced from the first surface.

Aspect 86. The apparatus of any of aspects 56 to 85, wherein vibrating the one or more vibratory transducers comprises vibrating the one or more vibratory transducers at a vibration frequency, wherein the vibration frequency is between 100 Hz and 100 kHz, preferably between 200 Hz and 10 kHz, more preferably between 300 Hz and 5 kHz, and more preferably between 500 Hz and 2 kHz, even more preferably at or approximately at 1 KHz. Aspect 87. The apparatus of any of aspects 56 to 86, wherein the first and second surfaces are located on the same vibratory transducer.

Aspect 88. The apparatus of aspect 87, wherein the first and second surfaces are located on different vibratory transducers configured to vibrate at the same frequency.

Aspect 89. The apparatus of any of aspects 56 to 88, wherein the first and second surfaces are configured to vibrate in phase with each other.

Aspect 90. The apparatus of any of aspects 56 to 89, wherein the first surface is configured to vibrate at a phase offset relative to the second surface.

Aspect 91. The apparatus of any of aspects 56 to 90, wherein the first and second waves are shear waves.

Aspect 92. The apparatus of any of aspects 56 to 91, wherein determining a measurement of a material property of the viscoelastic fluid comprises determining a loss factor or a Q factor of the vibration of the one or more vibratory transducers in the viscoelastic fluid.

Aspect 93. The apparatus of aspect 92, wherein the determined loss factor or Q factor is a monotonic function of the viscosity or storage modulus of the viscoelastic fluid.

Aspect 94. A non-transitory computer-readable medium having instructions stored thereon that, when executed by one or more processors of a system comprising one or more vibratory transducers, cause the one or more processors to perform a method according to any of aspects 1 to 55.

Aspect 95. An apparatus as described herein or substantially as described herein with reference to the description, claims and drawings, wherein, during vibration of the one or more vibratory transducers, the first and second waves do not combine with each other to provide a net constructive or destructive interference at one or both of the first and second surfaces.

Aspect 96. A method as described herein or substantially as described herein with reference to the description, claims and drawings, wherein, during vibration of the one or more vibratory transducers, the first and second waves do not combine with each other to provide a net constructive or destructive interference at one or both of the first and second surfaces.

The previous description of the disclosed embodiments is provided to enable any person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as defined by the following claims.

1. A method of measuring a material property of a viscoelastic fluid using one or more vibratory transducers, the method comprising:
vibrating one or more vibratory transducers in the viscoelastic fluid to generate a first wave propagating from a first surface of the one or more vibratory transducers and a second wave propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces; and
determining a material property of the viscoelastic fluid based on the vibrating of the one or more vibratory transducers in the viscoelastic fluid.

2. The method of claim 1, wherein the first and second waves combine with each other to provide a net destructive interference at one or both of the first and second surfaces.

3. The method of claim 1 or claim 2, wherein the first and second waves are shear waves.

4. The method of any of claims 1 to 3, wherein determining a measurement of a material property of the viscoelastic fluid comprises determining a loss factor or a Q factor of the vibration of the one or more vibratory transducers in the viscoelastic fluid.

5. The method of claim 4, wherein the determined loss factor or Q factor is a monotonic function of the viscosity or storage modulus of the viscoelastic fluid.

6. The method of any of claims 1 to 5, wherein tan Δ of the viscoelastic fluid is less than 1, wherein tan Δ is the loss tangent of the viscoelastic fluid.

7. The method of any of claims 1 to 7, wherein one or both of the first and second surfaces is curved or comprises a curved portion.

8. The method of claim 7, wherein one or both of the first and second surfaces is concave or comprises a concave portion, wherein the first wave focuses at a focal distance from the first surface.

9. The method of claim 8, wherein the second surface is located further from the first surface than the focal distance is from the first surface.

10. The method of claim 8 or claim 9, wherein a concave portion of the first surface comprises a first region of the concave portion and a second region of the concave portion that is configured to vibrate out of phase with the first region of the concave portion to generate a wave that is out of phase with a wave generated from the first region of the concave portion.

11. The method of any of claims 1 to 10, wherein one or both of the first and second surfaces comprise an elongate member, wherein vibrating the one or more vibratory transducers in the viscoelastic fluid comprises vibrating the one or more vibratory transducers through or about a vibrational axis of the one or more vibratory transducers, wherein the vibrational axis is not colinear with the elongate member.

12. The method of claim 11, wherein vibrating the one or more vibratory transducers in the viscoelastic fluid comprises vibrating the one or more vibratory transducers torsionally about a common vibrational axis, wherein one or both of the first and second surfaces comprise an elongate member configured in the form of a ring, wherein an axis through the centre of the ring is colinear with the common vibrational axis.

13. The method of claim 12, wherein the first surface comprises an elongate member configured in the form of a ring that is connected to the second surface by one or more support members that offset the first surface from the second surface.

14. The method of claim 12 or claim 13, wherein the second surface comprises a concave portion configured to generate shear waves under torsional vibration about the common vibrational axis, the generated shear waves focusing toward the first surface.

15. The method of claim 11, wherein the one or more vibratory transducers comprise a shaft having a longitudinal axis extending along the shaft and a plurality of elongate members extending outward from the longitudinal axis and spaced from each other, wherein vibrating the one or more vibratory transducers comprises torsionally vibrating the shaft about the longitudinal axis.

16. The method of any of claims 1 to 15, wherein the first and second waves are shear waves and wherein one or both of the first and second surfaces comprise an elongate member characterised by a width, a half width that is equal to half of the width, and a length that is greater than the width, wherein the half width is less than a propagation depth of a shear wave in the fluid at the vibration frequency.

17. The method of any of claims 1 to 16, wherein vibrating the one or more vibratory transducers comprises vibrating the one or more vibratory transducers at a vibration frequency, wherein the vibration frequency is between 500 Hz and 2 kHz.

18. An apparatus for measuring a material property of a viscoelastic fluid using one or more vibratory transducers, the apparatus comprising:
one or more vibratory transducers, the one or more vibratory transducers comprising a first surface and a second surface;
means for vibrating the one or more vibratory transducers such that, when vibrated in a viscoelastic fluid, a first wave is generated propagating from a first surface of the one or more vibratory transducers and a second wave is generated propagating from a second surface of the one or more vibratory transducers, wherein the first and second surfaces are spaced and oriented relative to each other such that, during vibration of the one or more vibratory transducers, the first and second waves combine with each other to provide a net constructive or destructive interference at one or both of the first and second surfaces; and
means for determining a material property of the viscoelastic fluid based on the vibrating of the one or more vibratory transducers in the viscoelastic fluid including the net constructive or destructive interference.

19. The apparatus of claim 18, wherein the first surface comprises: an elongate member, a flat surface, and/or a concave portion configured to generate waves under vibration that focus toward the second surface, and wherein the second surface comprises: an elongate member, a flat surface, and/or a concave portion configured to generate waves under vibration that focus toward the first surface.

20. The apparatus of claim 18 or claim 19, wherein one or both of the first and second surfaces comprise an elongate member, wherein vibrating the one or more vibratory transducers in the viscoelastic fluid comprises vibrating the one or more vibratory transducers through or about a vibrational axis of the one or more vibratory transducers, wherein the vibrational axis is not colinear with the elongate member.

21. The apparatus of claim 20, wherein vibrating the one or more vibratory transducers in the viscoelastic fluid comprises vibrating the one or more vibratory transducers torsionally about a common vibrational axis, wherein one or both of the first and second surfaces comprise an elongate member configured in the form of a ring, wherein an axis through the centre of the ring is colinear with the common vibrational axis.

22. The method of claim 21, wherein the first surface comprises an elongate member configured in the form of a ring that is connected to the second surface by one or more support members that offset the first surface from the second surface.

23. The apparatus of claim 21 or claim 22, wherein the second surface comprises a concave portion configured to generate shear waves under torsional vibration about the common vibrational axis, the generated shear waves focusing toward the first surface.

24. The apparatus of claim 20, wherein the one or more vibratory transducers comprise a shaft having a longitudinal axis extending along the shaft and a plurality of elongate members extending outward from the longitudinal axis and spaced from each other, wherein vibrating the one or more vibratory transducers comprises torsionally vibrating the shaft about the longitudinal axis.

25. The apparatus of any of claims 18 to 24, wherein one or both of the first and second surfaces comprise an elongate member characterised by a width, a half width that is equal to half of the width, and a length that is greater than the width, wherein the half width is less than a propagation depth of a shear wave in the fluid at the vibration frequency.

26. The apparatus of any of claims 18 to 25, wherein the first and second surfaces are located on the same vibratory transducer or on different vibratory transducers that are configured to vibrate at the same frequency.

27. The apparatus of any of claims 18 to 26, wherein the first and second surfaces are configured to vibrate in phase with each other or at a phase offset relative to each other.

28. A non-transitory computer-readable medium having instructions stored thereon that, when executed by one or more processors of a system comprising one or more vibratory transducers, cause the one or more processors to perform a method according to any of claims 1 to 17.
